(19) United States
(12) Patent Application Publication
(10) Pub. No.: US 2017/0166691 A1
(43) Pub. Date: Jun. 15, 2017

(54) METAL CHALCOGENIDE NANOSTRUCTURES

(71) Applicants: Jean-Paul LELLOUCHE, Ashdod (IL); Daniel RAICHMAN, Petah Tikva (IL); Rivka BEN ISHAY, Yakir (IL); Yifat HAREL, Holon (IL); Rina BEN-SHABAT-BINYAMINI, Tel-Aviv (IL); Hagit SADE, Ramat-Gan (IL)

(72) Inventors: Jean-Paul LELLOUCHE, Ashdod (IL); Daniel RAICHMAN, Petah Tikva (IL); Rivka BEN ISHAY, Yakir (IL); Yifat HAREL, Holon (IL); Rina BEN-SHABAT-BINYAMINI, Tel-Aviv (IL); Hagit SADE, Ramat-Gan (IL)

(21) Appl. No.: 15/376,924

(22) Filed: Dec. 13, 2016

Related U.S. Application Data
(60) Provisional application No. 62/266,627, filed on Dec. 13, 2015.

(51) Int. Cl. C08G 61/12; C07H 5/10

(52) U.S. Cl. CPC C08G 61/126; C07H 5/10; C08G 2261/516; C08G 2261/3223; C08G 2261/3247; C08G 2261/124; C08G 2261/43

(57) ABSTRACT

A hybrid inorganic-organic composite comprising a nanostructure of a metal chalcogenide and at least one compound comprising an organic cyclic moiety, wherein the compound is linked to the nanostructure, is provided. A nanotube of metal chalcogenide (e.g., metal dichalcogenide) nanostructure having attached to at least one surface thereof one or more selected chemically reactive nanoparticles is also provided.

This application claims the benefit of priority from U.S. Provisional Patent Application No. 62/266,627, filed on Dec. 13, 2015. The content of the above document is incorporated by reference in its entirety as if fully set forth herein.

### FIELD OF THE INVENTION

The present invention, in some embodiments thereof, relates to metal chalcogenide nanostructures.

### BACKGROUND OF THE INVENTION

Inorganic transition metal dichalcogenide materials, such as tungsten disulfides (WS₂), are of interest to the scientific community because of their unique layered structure and functional properties, with nanoparticles (NPs) tending to exhibit a different set of properties compared to the bulk forms. These metal di-chalcogenide nanomaterials have emerged as one of the most promising classes of nanomaterials since the discovery of carbon nanotubes (CNTs). As with early research in the field of CNTs, a number of potential applications have been proposed such as for energy storage, field effect transistors, nanocomposite coatings, battery anodes, light-emitting diodes, self-lubricating medical devices, and high-performance lubricants. In addition, the outstanding shock absorbing ability of WS₂NPs holds potential for new impact and shock resistant materials.

Hydrophobic inorganic nanotubes of tungsten disulfide (referred to hereinthroughout as “INT-WS₂”, or “WS₂-INTs”, interchangeably) are insoluble in common solvents and practically inert, hindering their usefulness in both research and commercial applications. The covalent attachment of functional species onto the surface of INT-WS₂ is a critical first step in realizing the potential that INT-WS₂ offer for high-performance materials and products. Although a few attempts have been reported regarding preparing modified nanotubes, only a limited range of surface functionalities is possible with the methods currently known in the art.

Raichman et al. (Inorganics 2014, 2(3), 455-467) disclose a design of a methodology used to identify and optimize factors that influence the degree of functionalization (polycarboxylation) of INT-WS₂ via a modified acidic Vilsmeier-Haack reagent.

Raichman et al. (Nano Research 2014, 8, 5, 1454-63) disclose a versatile method, based on a modified, highly electrophilic acidic Vilsmeier-Haack reagent, to produce covalently bonded, polycarboxylated functional WS₂ nanotubes that are dispersible in polar liquids, including water.

### SUMMARY OF THE INVENTION

The present invention provides, in some embodiments thereof, inorganic transition metal chalcogenide materials, particularly in the nano-sized form. The invention further provides surface functionalization/interfacial chemistries for providing the inorganic nanomaterials and use thereof.

According to some embodiments of the present invention, there is provided a hybrid inorganic-organic composite comprising a nanostructure of a metal chalcogenide and at least one compound comprising an organic cyclic moiety, wherein the compound is linked to the nanostructure.

In some embodiments, the cyclic moiety is a heterocyclic moiety.

In some embodiments, the metal chalcogenide is metal dichalcogenide.

In some embodiments, the compound is covalently linked to the nanostructure. In some embodiments, the compound is covalently linked to the nanotube via a linker.

In some embodiments, the nanostructure is in the form of a nanotube.

In some embodiments, the nanostructure is in the form of fullerene.

In some embodiments, the linker is an organic linker. In some embodiments, the linker is an inorganic linker. In some embodiments, the linker comprises a carbonyl group.

In some embodiments, the compound has the formula (I):

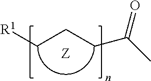

such that the hybrid composite has the structure of Formula II:

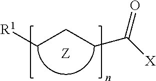

wherein:
Z represents a cyclic or heterocylic moiety;
n is an integer of from 1 to 100;
X represents a metal chalcogenide or a metal dichalcogenide selected from the group consisting of: TiS₂, TiSe₂, TiTe₂, WS₂, WSe₂, WTe₂, MoS₂, MoSe₂, MoTe₂, SnS₂, SnSe₂, SnTe₂, RuS₂, RuSe₂, RuTe₂, GaS, GaSe, GaTe, InS, InSe, HfS₂, ZrS₂, VS₂, ReS₂ or NbS₂; and
R₁ represents or comprises hydrogen, any polymer and/or organic moiety selected from the group consisting of: hydrogen, alkyl, cycloalkyl, aryl, heterocyclic, heteroaryl, alkoxy, hydroxy, thiohydroxy, thioalkoxy, aryloxy, thioaryloxy, aniline, amino, nitro, halo, trihalomethyl, cyano, amide, carboxyl, sulfonyl, sulfoxy, sulfinyl, sulfonamide, and a saccharide.

In some embodiments, Z or R₁ are at least partially enzymatically-cleavable.

In some embodiments, the compound comprising a cyclic moiety is covalently linked to the nanostructure via S or Se atom linkage.

In some embodiments, the cyclic moiety is a polycyclic moiety.

In some embodiments, the disclosed composite is in the form of Formula (III):

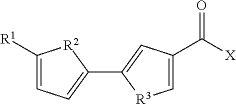

wherein R² and R³ are each, independently, selected from the elements S and O.

In some embodiments, R² and R³ represent S atom.

In some embodiments, R¹ is represented by Formula (IV)

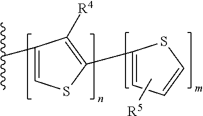

wherein n and m are each independently an integer of from 0 to 100;
R⁴ comprises or is selected from the group consisting of: hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, and a saccharide; and
R⁵ comprises or is selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, a saccharide or is a fused ring.

In some embodiments, the disclosed composite has a structure represented by Formula V:

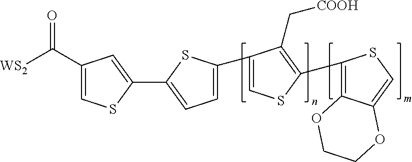

In some embodiments, the disclosed composite is in the form of a core-shell, wherein the core is or comprises a plurality of the metal chalcogenide and the shell is or comprises a plurality of the cyclic moiety. In some embodiments, an outside surface of the shell is characterized by a layer selected from a polyCOOH, polyOH and chemical derivative thereof. In some embodiments, the cyclic moiety is or comprises a saccharide-related moiety and wherein the nanostructure is attached to an anomeric carbon of the saccharide moiety.

In some embodiments, the saccharide moiety is a monosaccharide.

In some embodiments, the saccharide moiety is a polysaccharide-based polymeric species.

According to some embodiments of the present invention, there is provided a process for producing the hybrid composite of claim 16, comprising the steps of:

mixing bithiophene or a derivative thereof and a dehydrating agent is selected from POCl₃, PCl₃, SOCl₂, SO₂Cl₂, SO₃, PCl₅, P₂O₅, VOCl₃, AlCl₃, TiCl₄, acetic anhydride, trifluoromethanesulfonic anhydride thereby synthesizing 2,2′-bithiophene-5-carboxaldehyde; and adding to the 2,2′-bithiophene-5-carboxaldehyde a nanostructure of metal chalcogenide or a metal dichalcogenide, thereby producing the hybrid material.

According to some embodiments of the present invention, there is provided a nanotube of metal chalcogenide nanostructure having attached onto at least one surface thereof one or more selected chemically reactive nanoparticles.

In some embodiments, the chemically reactive nanoparticles are selected from magnetically responsive inorganic nanotubes and fullerene nanoparticles. In some embodiments, the chemically reactive nanoparticles are Fe₂O₃ nanoparticles. In some embodiments, the Fe₂O₃ nanoparticles are metal cation or complex-doped maghemite γ-Fe₂O₃.

In some embodiments, the disclosed nanotube further comprises cerium cations.

In some embodiments, the disclosed nanotube further comprises complexes.

In some embodiments, the disclosed is in the form of a core-shell, wherein the core comprises a metal chalcogenide and the shell comprises the metal cation or complex-doped maghemite γ-Fe₂O₃, and optionally further comprises cerium ions.

According to some embodiments of the present invention, there is provided an article comprising the disclosed hybrid composite and/or the disclosed nanotube. In some embodiments, the hybrid composite is in the form of coating of at least one surface of the article.

In some embodiments, the article is for use in medicine.

In some embodiments, the article is selected from: mechanically reinforced polymeric 2D coatings, 3D matrices, 3D fibers, 3D polymer printing structures, flame-retardant 2D coatings, mechanically reinforced filtration membranes, lubricant hybrid systems, non-toxic biological implants, bone tissue engineering systems, mobile functioning human joints, tissue-penetrating devices, drug delivery hybrid systems, conductive electrode components for batteries, super-capacitors, and diagnostic/sensing devices, and field effect transistor FET.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-I present schematic representations of some embodiments of the present invention: formation of INT-WS₂-bithiophene—Intermediate nanomaterial before polythiophene growth (LPP method) from nucleophilized Th-based inorganic nanotubes (INT)-WS₂ (three steps) (FIG. 1A, referred to as “scheme 1”); LPP polymerization of polythiophene acetic acid/polyethylenedioxythiophene (PTAA/PEDOT) onto the Th-functionalized INT/IF WS₂ surface (FIG. 1B, referred to as “scheme 2”) (IF: inorganic fullerene); Chemical structure of acidic (polyCOOH) polyThA (polythiophene acetic acid) (FIG. 1C, referred to as “scheme 3”); Two-step fabrication of dual phase polyTh/INTs via (i) coupling with a thiophene linker and (ii) in situ oxidatively polymerizing thiophene monomers (FIG. 1D, referred to as “scheme 4”); Fabrication of polyOH-functionalized INT-WS₂ nanomaterials using S-glycosidation chemistry for surface derivatization/growth of a polysaccharide/poolyheteroaromatic shell (FIG. 1E, referred to as “scheme 5”); Schematic description of (OEt)₃Si(CH₂)₃NH₂ (APTES) reaction with hydroxyl groups (FIG. 1F, referred to as “scheme 6”); Schematic preparation of CAN-γ-Fe₂O₃ nanoparticles (CAN: ceric ammonium nitrate) (CAN-γ-Fe₂O₃ NPs) showing the Ce^{3/4+} cation/complex-doping phase (FIG. 1G, referred to as “scheme 7”); Isolation of WS₂-maghemite nanoparticles from the solution (FIG. 1H, referred to as “scheme 8”); Isolation of INTs/IFs-CAN-γ-Fe₂O₃ NPs composites from experiment solutions via magnet attractive forces apart from starting CAN-maghemite NPs and non-magnetic INTs/IFs that were not attached by magnetic CAN-γ-Fe₂O₃ nanoparticles (FIG. 1I, referred to as “scheme 9”).

FIG. 2 presents a graph showing Raman spectra of INTs/IF-WS₂—PTAA.

FIGS. 3A-E present high-resolution transmission electron microscopy (HR-TEM) images (FIG. 3A-C) and Cryo-TEM images (FIGS. 3D & 3E) of INTs-WS₂—PTAA Cryo-TEM and HR-TEM images showing poly-thiophene acetic acid coating with a width of around 11 nm.

FIGS. 4A-E present HR-TEM images (FIG. 3A-C) and Cryo-TEM images (FIGS. 3D & 3E) of IFs-WS₂—PTAA.

FIG. 5 presents electron paramagnetic resonance (EPR) spectra of WS₂, WS₂-bithiophene and WS₂—PTAA (INTs (left panel and IFs (right panel)).

FIG. 6 presents thermogravimetric (TGA) spectra of WS₂ INTs vs WS₂-bithiophene and WS₂—PTAA.

FIG. 7 presents TEM images of the core-shell structure of the polythiophene-INTs composites (left panel: scale bar is 100 nm; right panel: scale bar is 50 nm).

FIG. 8 presents TGA graphs of commercial starting INTs (dashed line) and polyThA INTs showing % of weight losses versus increasing temperatures.

FIG. 9 presents Fourier-transform infrared (FT-IR) spectra of commercial INTs, of polyCOOH-INTs, of Th-linker-INTs, and of polyThA INTs composites (from upper to lower panels (graphs), respectively).

FIG. 10 presents a Raman spectrum of polyThA INTs composites.

FIG. 11 presents HRSEM images of synthesized maghemite (γ-Fe₂O₃) nanoparticles onto WS₂ INT (left panel: scale bar is 300 nm, right panel: on the right 1 m).

FIG. 12 presents a spectrum showing elemental analysis of maghemite (γ-Fe₂O₃) nanoparticles deposited onto WS₂ INT using high resolution scanning electron microscopes and energy-dispersive X-ray spectroscopy HRSEM-EDX.

FIGS. 13A-F present TEM images of untreated WS₂-INTs (FIG. 13A), Ox-INT-WS₂ treated with H₂O₂(FIG. 13B), K₂Cr₂O₇(FIG. 13C), and Fenton (FIG. 13D), and after APTES derivatization: APTES-K₂Cr₂O₇— WS₂-INTs (FIG. 13E) and APTES-MCPBA-WS₂—INTs (FIG. 13F) (Ox: oxidized).

FIG. 14 presents graphs showing TGA and inset DTG plots of the untreated-WS₂-INTs, Ox-CAN-INT-WS₂, and APTES-CAN-INT-WS₂ under nitrogen.

FIG. 15 presents FTIR spectra of the untreated INT-WS₂, Ox-Fenton-INT-WS₂, and APTES-Fenton-INT-WS₂.

FIG. 16 presents graphs showing TGA (top) and DTG (bottom) curves of samples A-G (see Table 9) and untreated WS₂ NTs. Y-axis represents % residual weight.

FIGS. 17A-H present electron micrographs of samples A-G (according to Table 9 below, and FIG. 16, respectively) and further present non-coated WS₂ NTs (upper left panel, Figure H).

FIG. 18 presents HRTEM images (top panel) and Energy-dispersive X-ray spectroscopy (EDS) spectra (bottom) taken from different points (1-4) in sample G (according to Table 9 below, FIG. 16 and FIG. 17G).

FIG. 19 presents scanning transmission electron microscope (STEM; bar is 250 nm) images (left panel) and EDS line scanning profile (right panel) of tungsten, sulfur and carbon elements in a coated modified nanotube sample (Sample F according to Table 9, FIG. 16 and FIG. 17F).

FIG. 20 presents HRSEM images of samples B, D, F, G, (as denoted respectively by the letters “B”, “D”, “F”, and “G” in FIG. 20), and untreated WS₂NTs (denoted in FIG. 20 as “WS₂ NTs”).

FIG. 21 presents Raman spectra of samples A-G (according to Table 9, FIGS. 16 and 17A-G) and of untreated WS₂ NTs.

FIGS. 22A-D present attenuated total reflectance (ATR)-FTIR transmittance spectra of samples A-G and untreated WS₂ NTs. Plots a and b (FIGS. 22A and 22B, respectively) show the series of samples in the ranges of 400-1900 and 2600-3700, respectively. Plots c and d (FIGS. 22C and 22D, respectively) focus on sample F.

FIG. 23 presents a bar graph C (Zeta) potential values in mV for samples A-G (according to Table 9, FIGS. 16 and 17A-G) and for untreated WS₂ NTs, before and after APTES attachment.

FIG. 24 presents a bar graph showing Zeta Potential (mV) of different ratios of INTs to CAN-maghemite NPs at various time duration of attachment reaction (3, 6, 18 hours).

FIG. 25 presents HR-TEM image of INTs/IFs-CAN-γ-Fe₂O₃ NP composites (left panel; scale bar is 5 nm) and selected area electron diffraction (SAED) RX diffraction of NPs-coated WS₂ INTs (inset left panel), and elemental mapping of CAN-γ-Fe₂O₃ NPs onto WS₂ INTs via HRSEM (right panel; W-green & Fe-red).

FIG. 26 presents HRSEM images of CAN-γ-Fe₂O₃ NPs attached onto WS₂-INTs at different scale bars (left panel: 300 nm; middle panel: 11 μm, and right panel: 500 nm).

FIG. 27 presents HRTEM (left panel; scale bar is 20 nm) and TEM (right panel) images of CAN-γ-Fe₂O₃ attached onto WS₂-INTs.

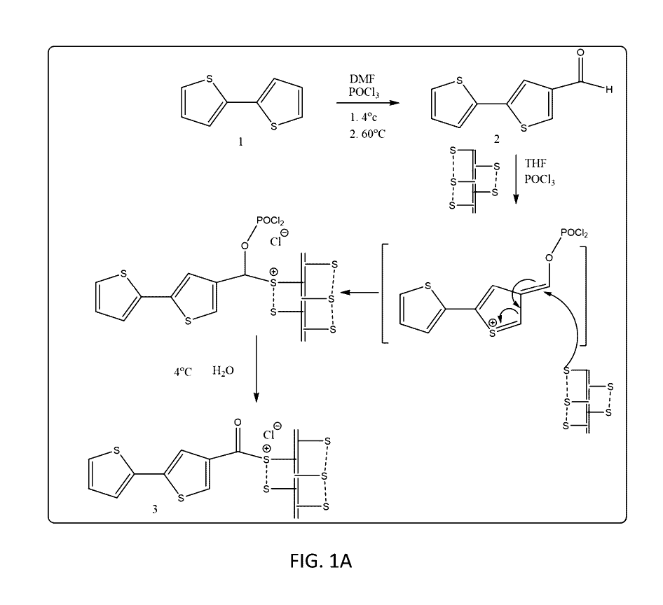

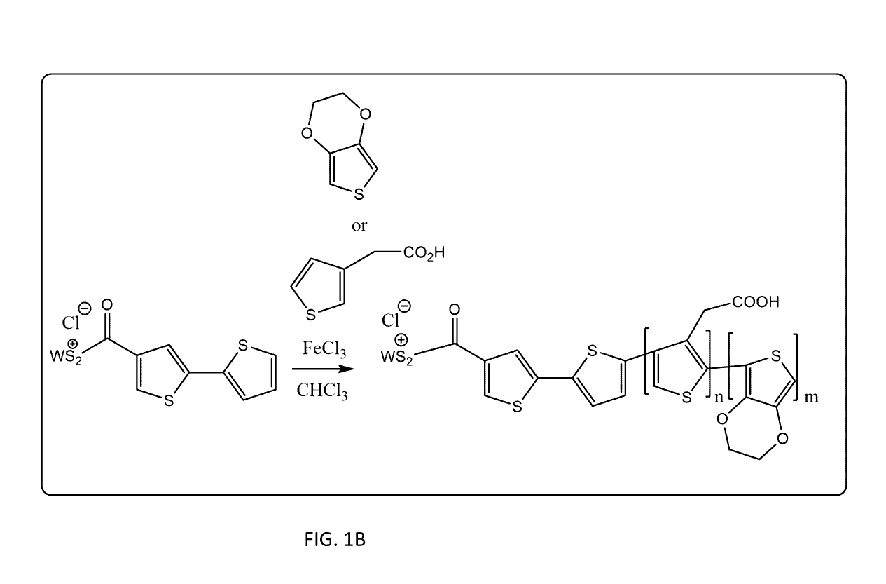

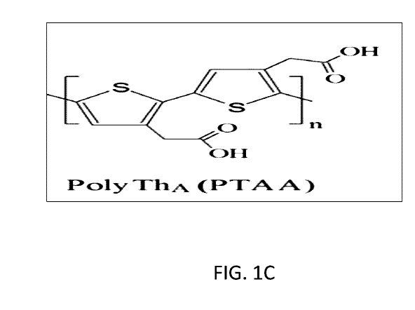

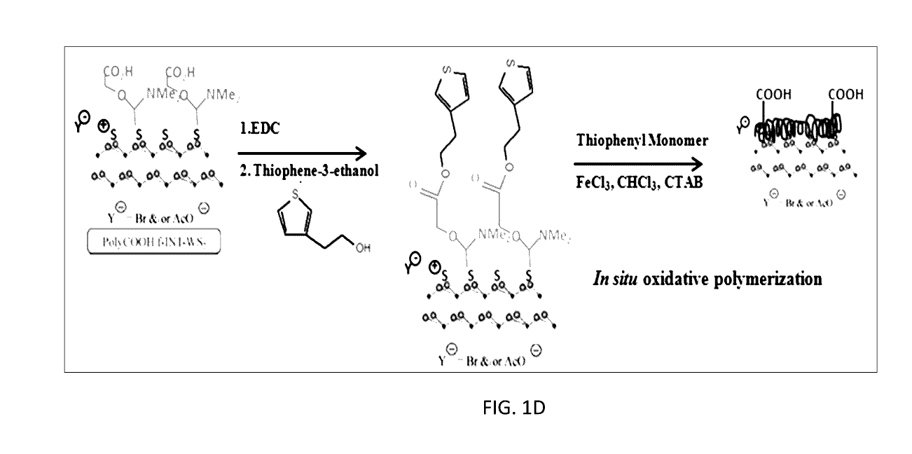

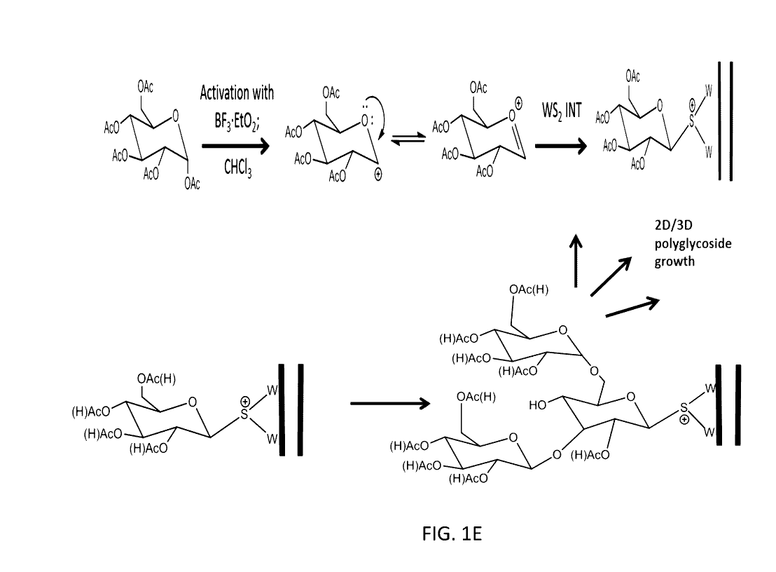

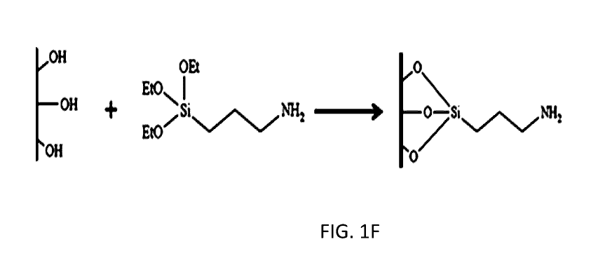

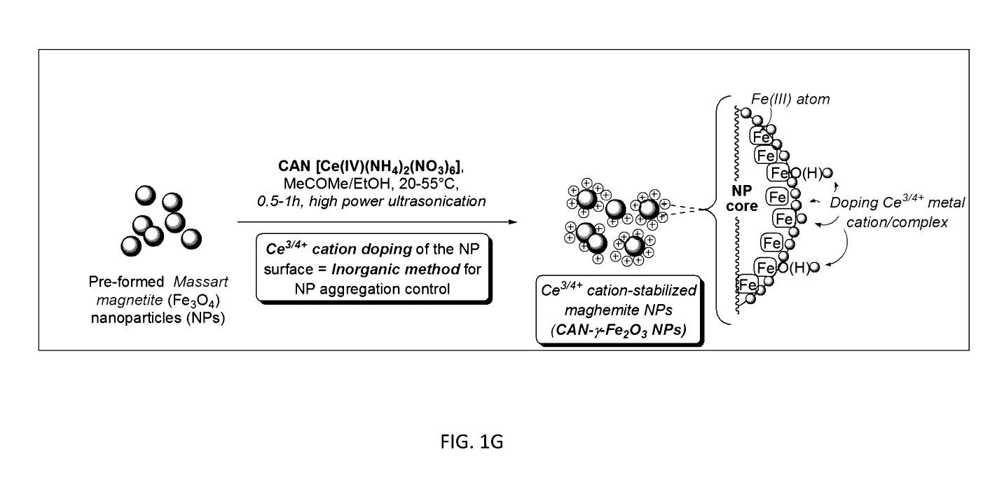

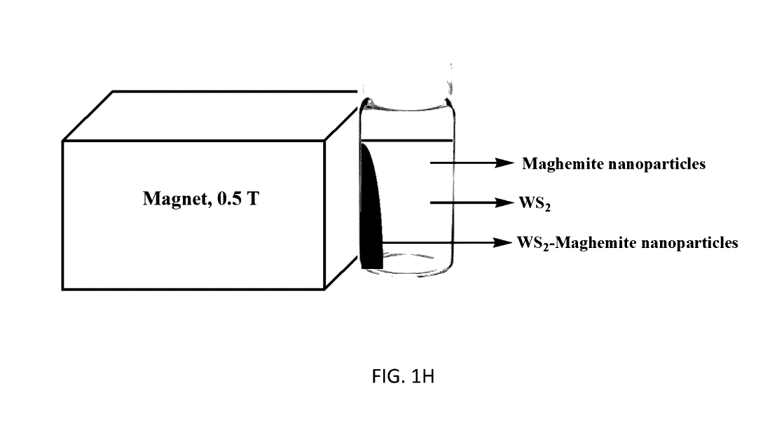

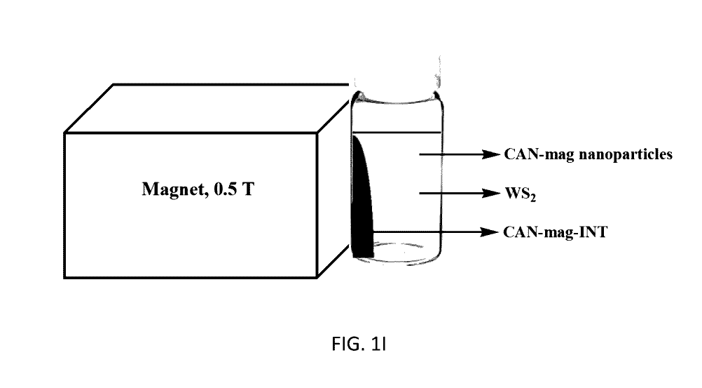

### DETAILED DESCRIPTION OF THE INVENTION

The present invention provides, in some embodiments thereof, inorganic transition metal chalcogenide materials, particularly in the nano-sized form. The invention further provides surface functionalization/interfacial chemistries for providing the inorganic nanomaterials and use thereof.

According to an aspect of some embodiments of the present invention, there is provided an inorganic-organic composite comprising a nanostructure of a metal chalcogenide and a compound comprising an organic cyclic (e.g., heterocyclic) moiety.

In some embodiments, the compound is linked to the nanostructure. In some embodiments, the compound is physically linked to the nanostructure. In some embodiments, the compound is chemically linked to the nanostructure.

In some embodiments, the inorganic-organic composite is a hybrid inorganic-organic composite.

The term “hybrid inorganic-organic composite” may refer to a material having chemical bonds among inorganic and organic unit(s) of a composite material, forming a matrix throughout the material itself, as opposed to a mixture of discrete chemical compounds, for example.

As used herein and in the art, the term “chemical bond” and its grammatical variations refer to a coupling of two or more atoms based on an attractive interaction thereof. “Chemical bond” may refer to, without being limited thereto, Van der Waals, dipole, covalent or hydrogen bonds. That is a chemical bond may also be covalent or ionic bond(s) between the organic and inorganic substances (i.e. the compound and the nanostructure, respectively).

In some embodiments, the compound is covalently linked to the nanostructure. By “covalently linked” it is meant to refer to any chemical bond other than a purely ionic bond.

In some embodiments, the nanostructure is in the form of a nanotube or a fullerene nanoparticle.

As used herein, the term “metal chalcogenide”, unless otherwise specifically referred to a specific metal species, refers to a compound of a metal with sulfur, selenium or tellurium or polonium. Exemplary metals include Ga, In, Ti, Sn, Pb, Bi, Zn, Cd, Hg, Cu, Ag, Au, Ni, Pd, Pt, Co, Rh, Ir, Fe, Ru, Os, Mn, Tc, Re, Cr, Mo, W, V, Nb, Ta, Ti, Zr, Hf, Sc and Y. In some embodiments, “metal chalcogenide” refers to metal dichalcogenide.

In one embodiment, the metal chalcogenide is TiS₂. In some embodiments, the metal chalcogenide is TiSe₂. In some embodiments, the metal chalcogenide is TiTe₂. In some embodiments, the metal chalcogenide is WSe₂. In some embodiments, the metal chalcogenide is WTe₂. In some embodiments, the metal chalcogenide is MoS₂. In some embodiments, the metal chalcogenide is MoSe₂. In some embodiments, the metal chalcogenide is MoTe₂. In some embodiments, the metal chalcogenide is SnS₂. In some embodiments, the metal chalcogenide is SnSe₂. In some embodiments, the metal chalcogenide is SnTe₂. In some embodiments, the metal chalcogenide is RuS₂. In some embodiments, the metal chalcogenide is RuSe₂. In some embodiments, the metal chalcogenide is RuTe₂. In some embodiments, the metal chalcogenide is GaS. In some embodiments, the metal chalcogenide is GaSe. In some embodiments, the metal chalcogenide is GaTe. In some embodiments, the metal chalcogenide is InS. In some embodiments, the metal chalcogenide is InSe. In some embodiments, the metal chalcogenide is HfS₂. In some embodiments, the metal chalcogenide is ZrS₂. In some embodiments, the metal chalcogenide is VS₂. In some embodiments, the metal chalcogenide is ReS₂. In some embodiments, the metal chalcogenide is NbS₂. In exemplary embodiments, the metal chalcogenide is WS₂.

In some embodiments, the term “unit(s)” refers to inorganic organic repeat unit(s) (e.g., comprising S—W—S bonds between repeat units), organic repeat unit(s) (e.g., comprising C—C bonds between repeat unit(s)), or mixed organic-inorganic repeat unit(s) (e.g., comprising both C—C and S—W—S bonds between repeat unit(s)).

Hereinthroughout, the term “nanostructure”, describes a structure featuring a size of at least one dimension thereof (e.g., diameter, length) that ranges from about 1 nanometer to 1000 nanometers.

In some embodiments, the size of the nanostructure described herein represents an average size of a plurality of nanostructure composites.

In some embodiments, the average size (e.g., diameter, length) ranges from about 1 nanometer to 1000 nanometers. In some embodiments, the average size (e.g., diameter, length) ranges from about 1 nanometer to 500 nanometers. In some embodiments, the average size ranges from about 1 nanometer to about 300 nanometers. In some embodiments, the average size ranges from about 1 nanometer to about 200 nanometers. In some embodiments, the average size ranges from about 1 nanometer to about 100 nanometers. In some embodiments, the average size ranges from about 1 nanometer to 50 nanometers, and in some embodiments, it is smaller than 35 nm.

The structure can be generally shaped as a sphere, a rod, a cylinder, a ribbon, a sponge, a tube, a fullerene-like and any other shape, or can be in a form of a cluster of any of these shapes, or can comprises a mixture of one or more shapes.

In some embodiments, the term “fullerene-like” refers to inorganic metal chalcogenide structures having one layer or nested layers which form as a closed cage which may encage a void (i.e., be hollowed) or a core or may form a stuffed nested layer structure, e.g., a structure containing a material other than the metal material precursor encaged within nested layers of the metal chalcogenide. In some embodiments, the term refers to structures selected from single and double layer inorganic fullerenes, nested layers inorganic fullerenes, stuffed inorganic fullerenes, single layer nested nanotubes, stuffed nanotubes, and inorganic supperlattice structures, i.e. having layers of two or more different metal chalcogenides, e.g., WS₂ and WSe₂.

In some embodiments, the metal chalcogenide nanostructure includes one or more layers of desired sizes and shapes, e.g., spheres, sphere-like, nanotubes and polyhedral shapes.

As used herein the term “moiety” describes a major portion of a first molecule which is covalently linked to another molecule and which retains its main structural features and/or activity. Thus, a “moiety” refers to a part of a molecule formed by conjugating the aforementioned first molecule to one or more other molecules, and represents that portion of the first molecule that is present in the conjugation product. For example, a carboxylic acid moiety is the R—C(═O)— portion of a R—C(═O)OH carboxylic acid molecule formed upon conjugating the latter to an amine group in a second molecule, to thereby obtain an amide. In another example, an alkyl moiety is the portion of an alkyl halide molecule formed upon a nucleophilic reaction between the alkyl halide and an electrophilic molecule.

In some embodiments, the compound is covalently linked to the nanostructure (e.g., nanotube) via a linker. In some embodiments, the linker is an organic linker. In some embodiments, the linker is an inorganic linker. In one embodiment, the organic linker is a single, straight chain linker. In some embodiments, the linker may also include branched chains. As used herein, the term “linker” refers to a bond, e.g., a covalent bond. In one embodiment, the organic linker comprises reactive group that forms a part of the linker.

As used herein, the phrase “reactive group” describes a chemical group that is capable of undergoing a chemical reaction that typically leads to a bond formation. Chemical reactions that lead to a bond formation include, for example, nucleophilic and electrophilic substitutions, nucleophilic and electrophilic addition reactions, alkylations, addition-elimination reactions, cycloaddition reactions, rearrangement reactions and any other known organic reactions that involve a functional group, as well as combinations thereof.

In some embodiments, the reactive group is selected suitable for undergoing a chemical reaction that leads to a bond formation with a complementary functionality.

The reactive group may optionally comprise a non-reactive portion (e.g., an alkyl) which may serve, for example, to attach a reactive portion of the reactive group to a moiety.

In exemplary embodiments, the linker comprises a carbonyl group.

In some embodiments, the term “inorganic linker” refers to an inorganic binding entity.

In some embodiments, the inorganic linker contains non-carbon atoms in its backbone structure. Thus, an inorganic linker may or may not include an organic group pendant on the backbone structure. Non-limiting examples of inorganic linker are a siloxane linker phosphazene (i.e., —(R)₂P═N—) groups, polysulfide groups (i.e., polysulfide salt or inorganic polysulfide), borazine (i.e., —B═N—) groups, and polystannane groups.

In some embodiments, the compound has the formula (I):

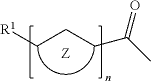

such that the hybrid composite has the structure of Formula II:

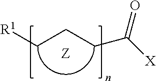

wherein:
Z represents a cyclic or heterocyclic moiety;
n is an integer of from 1 to 100, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100, including any integer value therebetween;
X represents a metal chalcogenide or a metal dichalcogenide; and
R₁ represents or comprises hydrogen, a polymer and/or organic moiety.

In one embodiment X represents TiS₂. In some embodiments X represents TiSe₂. In some embodiments X represents TiTe₂. In some embodiments X represents WSe₂. In some embodiments X represents WTe₂. In some embodiments X represents MoS₂. In some embodiments X represents MoSe₂. In some embodiments X represents MoTe₂. In some embodiments X represents SnS₂. In some embodiments X represents SnSe₂. In some embodiments X represents SnTe₂. In some embodiments X represents RuS₂. In some embodiments X represents RuSe₂. In some embodiments X represents RuTe₂. In some embodiments X represents GaS. In some embodiments X represents GaSe. In some embodiments X represents GaTe. In some embodiments X represents InS. In some embodiments X represents InSe. In some embodiments X represents HfS₂. In some embodiments X represents ZrS₂. In some embodiments X represents VS₂. In some embodiments X represents ReS₂. In some embodiments X represents NbS₂. In an exemplary embodiment X is WS₂.

In some embodiments, R¹ represents hydrogen. In some embodiments, R¹ represents an alkyl moiety. In some embodiments, R¹ represents a cycloalkyl moiety. In some embodiments, R¹ represents an aryl moiety. In some embodiments, R¹ represents a heterocyclic moiety. In some embodiments, R¹ represents a heteroaryl moiety. In some embodiments, R¹ represents an alkoxy moiety. In some embodiments, R¹ represents a hydroxy moiety. In some embodiments, R¹ represents a thiohydroxy moiety. In some embodiments, R¹ represents a thioalkoxy moiety. In some embodiments, R¹ represents an aryloxy moiety. In some embodiments, R¹ represents a thioaryloxy moiety. In some embodiments, R¹ represents an aniline moiety. In some embodiments, R¹ represents an amino moiety. In some embodiments, R¹ represents a nitro moiety. In some embodiments, R¹ represents a halo atom. In some embodiments, R¹ represents a trihalomethyl moiety. In some embodiments, R¹ represents a cyano moiety. In some embodiments, R¹ represents an amide moiety. In some embodiments, R¹ represents a carboxyl moiety. In some embodiments, R¹ represents a sulfonyl moiety. In some embodiments, R¹ represents a sulfoxy moiety. In some embodiments, R¹ represents a sulfinyl moiety. In some embodiments, R¹ represents a sulfonamide moiety. In some embodiments, R¹ represents a saccharide moiety. In some embodiments R₁ represents a polymer.

As used herein, the term “polymer” describes a substance, e.g., an organic substance, or alternatively an inorganic substance, composed of a plurality of repeating structural units (referred to interchangeably as backbone units or monomeric units) covalently connected to one another and forming the polymeric backbone of the polymer. Hence, the term “polymer” as used herein encompasses organic and inorganic polymers and further encompasses one or more of a homopolymer, a copolymer or a mixture thereof (e.g., a blend). The term “homopolymer” as used herein describes a polymer that is made up of one type of monomeric units and hence is composed of homogenic backbone units. The term “copolymer” as used herein describes a polymer that is made up of more than one type of monomeric units and hence is composed of heterogenic backbone units. The heterogenic backbone units can differ from one another by the pendant groups thereof.

For the sake of simplicity, the terms “polymer” and “polymeric backbone” as used hereinthroughout interchangeably, relate to both homopolymers, copolymers and mixtures thereof. In some embodiments, the disclosed composite is biodegradable.

In some embodiments, the disclosed composite is biostable. In some embodiments, the disclosed composite is biocleavable.

In some embodiments, the term “biostable”, as used in this context of embodiments of the invention, describes a compound or a polymer that remains intact under physiological conditions (e.g., is not degraded in vivo, and hence is non-biodegradable or non-biocleavable).

In some embodiments, the term “biodegradable” describes a substance which can decompose under physiological and/or environmental condition(s) into breakdown products. Such physiological and/or environmental conditions include, for example, hydrolysis (decomposition via hydrolytic cleavage), enzymatic catalysis (enzymatic degradation), and mechanical interactions. This term typically refers to substances that decompose under these conditions such that 50 weight percent of the substance decompose within a time period shorter than one year.

In some embodiments, the term “biodegradable” as used in the context of embodiments of the invention, also encompasses the term “bioresorbable”, which describes a substance that decomposes under physiological conditions to break down products that undergo bioresorption into the host-organism, namely, become metabolites of the biochemical systems of the host-organism.

In some embodiments, Z and/or R¹ represent moieties susceptible to chemical cleavage in predetermine conditions. In some embodiments, Z and/or R¹ represent moieties that are soluble, biocompatible and can be hydrolyzed, digested or otherwise broken or cleaved, for example in an intracellular environment. In some embodiments, Z and/or R¹ are at least partially enzymatically cleavable.

In some embodiments, the compound comprising a cyclic/polycyclic moiety is covalently linked to the nanostructure via chalcogenide (e.g., S, or Se) atom linkage.

In some embodiments, the compound comprising a cyclic/polycyclic moiety is or comprises an aromatic compound. In some embodiments, the compound comprising a cyclic/polycyclic moiety is electrically conductive.

In some embodiments, the hybrid composite is in the form of Formula (III)

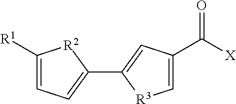

wherein R² and R³ are each, independently, selected from the elements S and O.

In some embodiments, at least one of R₂ and R₃ represent S. In some embodiments, both R₂ and R₃ represent S (see e.g., scheme 1).

In some embodiments, R¹ is represented by Formula (IV):

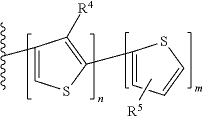

In some embodiments, n and m are each independently an integer of from 0 to 100.

In some embodiments, R⁴ is or comprises hydrogen. In some embodiments, R⁴ is or comprises alkyl. In some embodiments, R⁴ is or comprises a cycloalkyl moiety. In some embodiments, R⁴ is or comprises an aryl moiety. In some embodiments, R⁴ is or comprises a heteroalicyclic moiety. In some embodiments, R⁴ is or comprises a heteroaryl moiety. In some embodiments, R⁴ is or comprises a heteroaryl moiety. In some embodiments, R⁴ is or comprises an alkoxy moiety. In some embodiments, R⁴ is or comprises a hydroxyl moiety. In some embodiments, R⁴ is or comprises a thiohydroxy moiety. In some embodiments, R⁴ is or comprises a thioalkoxy moiety. In some embodiments, R⁴ is or comprises an aryloxy moiety. In some embodiments, R⁴ is or comprises a thioaryloxy moiety. In some embodiments, R⁴ is or comprises an amino moiety. In some embodiments, R⁴ is or comprises a nitro moiety. In some embodiments, R⁴ is or comprises a halo atom. In some embodiments, R⁴ is or comprises a trihalomethyl moiety. In some embodiments, R⁴ is or comprises a cyano moiety. In some embodiments, R⁴ is or comprises an amide moiety. In some embodiments, R⁴ is or comprises a carboxy moiety. In some embodiments, R⁴ is or comprises a sulfonyl moiety. In some embodiments, R⁴ is or comprises a sulfoxy moiety. In some embodiments, R⁴ is or comprises a sulfinyl moiety. In some embodiments, R⁴ is or comprises a sulfonamide moiety. In some embodiments, R⁴ is or comprises a saccharide moiety.

In some embodiments, R⁵ is or comprises hydrogen. In some embodiments, R⁵ is or comprises an alkyl moiety. In some embodiments, R⁵ is or comprises a cycloalkyl moiety. In some embodiments, R⁵ is or comprises an aryl moiety. In some embodiments, R⁵ is or comprises a heteroalicyclic moiety. In some embodiments, R⁵ is or comprises a heteroaryl moiety. In some embodiments, R⁵ is or comprises an alkoxy moiety. In some embodiments, R⁵ is or comprises a hydroxyl moiety. In some embodiments, R⁵ is or comprises a thiohydroxy moiety. In some embodiments, R⁵ is or comprises a thioalkoxy moiety. In some embodiments, R⁵ is or comprises an aryloxy moiety. In some embodiments, R⁵ is or comprises a thioaryloxy moiety. In some embodiments, R⁵ is or comprises an amino moiety. In some embodiments, R⁵ is or comprises a nitro moiety. In some embodiments, R⁵ is or comprises a halo atom. In some embodiments, R⁵ is or comprises a trihalomethyl moiety. In some embodiments, R⁵ is or comprises a cyano moiety. In some embodiments, R⁵ is or comprises an amide moiety. In some embodiments, R⁵ is or comprises a carboxy moiety. In some embodiments, R⁵ is or comprises a sulfonyl moiety. In some embodiments, R⁵ is or comprises a sulfoxy moiety. In some embodiments, R⁵ is or comprises a sulfinyl moiety. In some embodiments, R⁵ is or comprises a sulfonamide moiety. In some embodiments, R⁵ is or comprises a saccharide moiety. In some embodiments, R⁵ is or comprises a fused ring.

In some embodiments described herein, the hybrid composite is in the form of a salt.

In the context of some of the present embodiments, the salt of the hybrid composite described herein may optionally be an acid addition salt comprising at least one group or atom of the composite (e.g., S atom) which is in a positively charged form in combination with at least one counter-ion (e.g., Cl⁻) derived from the selected acid, that forms a salt.

In some embodiments, the hybrid composite has the structure represented by Formula V (see e.g., scheme 2):

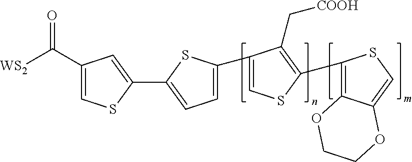

In exemplary embodiments, as further detailed in the Example section below, there is provided polymerization of polythiophene acetic acid/polyethylenedioxythiophene (PTAA/PEDOT) onto the Th-functionalized INT/IF WS₂ surface (IF—inorganic fullerene).

In some embodiments, n and m are each independently an integer of from 0 to 100.

In some embodiments, the disclosed composite is in the form of heterostructure. The term “heterostructure” as used herein means a structure in which materials having different compositions meet at interfaces.

A non-limiting example of heterostructure is the form of a core-shell structure.

In some embodiments, the hybrid composite is in the form of a core-shell. In some embodiments, the core is or comprises a plurality of the metal chalcogenide (e.g., metal dichalcogenide). In some embodiments, the shell is or comprises a plurality of the cyclic moiety.

The term “core-shell structure” generally refers to a solid material, wherein the solid material is a particulate material, and wherein individual particle(s) is characterized by containing at least two different types of materials which may be distinguished from one another by their composition and/or by their structure and/or by their placement within the particle, wherein one or more materials of a certain type are contained in the interior portion of the composite. The interior portion is designated by the term “core”, and one or more materials of a certain type which may be distinguished from the one or more materials contained in the interior portion are contained in the outer portion of the composite, thus forming the surface portion thereof. The outer portion comprising the surface is designated by the terms “shell” or “coating layer”.

In some embodiments, the core-shell structure is a closed structure.

The term “closed” as used herein, is a relative term with respect to the size, the shape and the particle or composition of two entities, namely an entity that defines an enclosure (the enclosing entity) and the entity that is being at least partially enclosed therein. In general, the term “closed” refers to a morphological state of an object which has discrete inner (e.g., the core) and outer surfaces which are substantially disconnected, wherein the inner surface constitutes the boundary of the enclosed area. The enclosed area may be at least partially secluded from the exterior area of space.

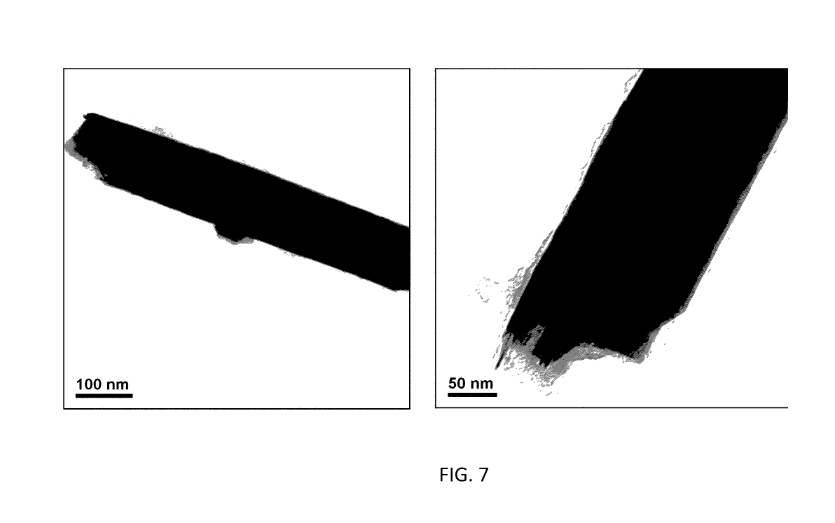

Reference is made to FIG. 7 showing core-shell structure of the polythiophene-INTs composites showing polythiophene acetic acid coating around the INTs (INT-inorganic nanotube).

In some embodiments, the core is or comprises a plurality of the metal chalcogenides (e.g., metal dichalcogenide). In some embodiments, the shell is or comprises a plurality of the cyclic moieties. In some embodiments, the shell (or the coating layer) has a thickness of e.g., 2 nm, 10 nm, 20 nm, 30 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 1 μm, 2 m, 5 m, 7 m, or 10 m, 20 μm, including any value and range therebetween.

In some embodiments, the shell (or the coating layer) has a thickness that ranges from e.g., 1 to 200 nm, 5 to 200 nm, 10 to 200 nm, 20 to 200 nm, 1 nm to 100 nm, or 5 nm to 50 nm, 200 nm to 1 μm, or 1 μm to 10 μm.

In some embodiments, an outside surface of the shell is characterized by a polyOH shell. In some embodiments, an outside surface of the shell is characterized by a polycarboxylated (polyCOOH) organic shell. In some embodiments, polyCOOH refers to surface-grafted polyCOOH organic shell.

In some embodiments, the “polyCOOH” further encompasses chemically derivatized polycarboxylate layer (see e.g., scheme 3).

By “chemically derivatized” it is meant to refer to a bearing a functional group, in addition to, or other than polyCOOH. The functional group may include, but is not limited to, the thiol (SH) or disulfide (—S—S—) group, or NH₂, OH and COOH. For example, in some embodiments, an outside surface of the shell is characterized by polyNH₂ shell. In some embodiments, an outside surface of the shell is characterized by a polyOH shell. In some embodiments, an outside surface of the shell is characterized by a polySH shell.

In some embodiments, the surface of the disclosed composite or the shell (e.g., “polyCOOH” or “polyOH”) is chemically derivatized by activating the shell with N-(3-dimethylaminopropyl)-N-ethyl-carbodiimide (EDC). In some embodiments, the shell is chemically derivatized by activating the shell with 1,1-Carbonyldiimidazole (CDI). Further exemplary embodiments are described in the Examples section below (e.g., Example 1) In some embodiments, the surface of the disclosed composite or the shell is chemically derivatized with silane groups, such as, for example and without limitation, 3-aminopropyltriethoxy silane (APTES), 3-(ethoxydimethylsilyl)propylamine, 3-(mercaptopropyl)triethoxysilane.

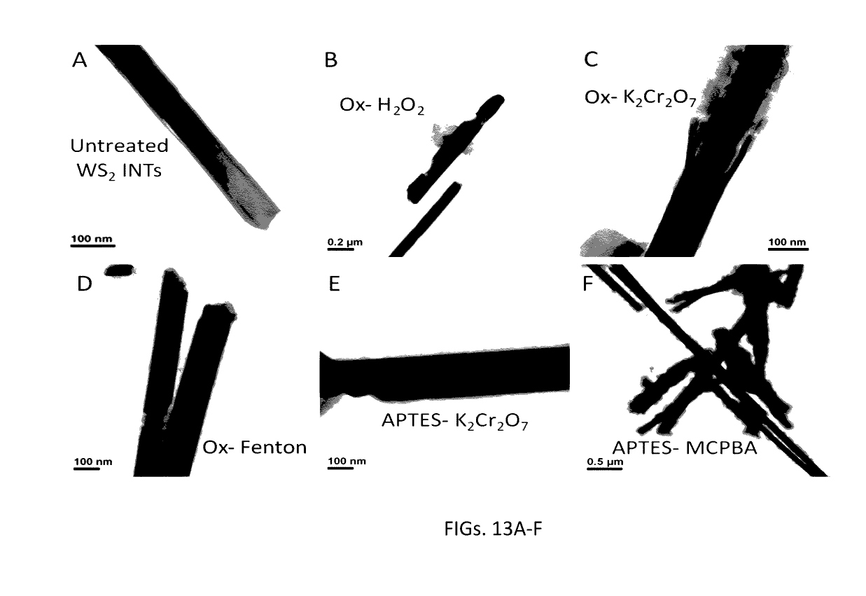

As described hereinbelow, in exemplary embodiments, INT-WS₂ may be oxidized. In some embodiments, the oxidation process comprises a subsequent derivatization with APTES. Reference is made to scheme 5 and to FIGS. 13 E and 13 F, and to the Examples section below.

In some embodiments, the surface of the disclosed composite or the shell further comprises a complex e.g., CAN (ceric ammonium nitrate). In exemplary embodiments, the disclosed composite is or comprises APTES-CAN-WS₂.

As used herein, the term “saccharide moiety” describes a moiety that comprises one or more saccharide units.

The term “saccharide moiety” may further refer to a portion of a saccharide molecule formed upon conjugating a second molecule thereto.

In exemplary embodiments of the invention, the saccharide moiety comprises one saccharide unit and the saccharide unit is a monosaccharide (see e.g., scheme 5).

The term “monosaccharide”, as used herein and in the art, may refer to a simple form of a sugar that consists of a single saccharide unit which cannot be further decomposed to smaller saccharide building blocks or moieties. Common non-limiting examples of monosaccharides include glucose (dextrose), fructose, galactose, mannose, and ribose.

Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms.

The above monosaccharides may encompass both D- and L-monosaccharides.

In some embodiments, the monosaccharide is a hexose or a hexose derivative. In some embodiments, the hexose is D-hexose. In alternative embodiments, the hexose is L-hexose.

In some embodiments, the monosaccharide may be a monosaccharide derivative, in which the saccharide unit comprises one or more substituents other than hydroxyls. Such derivative can be selected from, but is not limited to, ethers, esters, amides, acids, phosphates and amines. Amine derivatives include, for example, glucosamine, galactosamine, fructosamine and mannosamine. Amide derivatives include, for example, N-acetylated amine derivatives of saccharides (e.g., N-acetylglucosamine, N-acetylgalactosamine).

In some embodiments, the monosaccharide derivatives include mannose-6-phosphate, a phosphate derivative, and N-acetylneuraminic acid (a sialic acid), an acid and amide derivative. In some embodiments, the nanostructure is attached to an anomeric carbon of a saccharide moiety Reference is made to FIGS. 17A-G presenting TEM micrographs of polycarbohydrate coating (shell) on WS₂ nanotube. The polycarbohydrate coating (shell) may have a thickness sized e.g., 5 nm to 200 nm. For example, FIG. 17A shows a thickness of about 8 nm, FIG. 17B shows a thickness of about 8-11 nm, FIG. 17C shows a thickness of about 10-13 nm, FIG. 17D shows a thickness of about 20 nm, FIG. 17E shows a thickness of about 60-70 nm, FIG. 17F shows a thickness of about 90-110 nm, FIG. 17G shows a thickness of about 200 nm.

In some embodiments, the saccharide moiety is a polysaccharide-based polymeric species. In some embodiments, the saccharide moiety is a polysaccharide-based polymeric species forming a polyOH shell. In some embodiments, the saccharide moiety is a polysaccharide-based polymeric species forming a polyCOOH shell.

In some embodiments, the composite comprising a polyOH or polyCOOH shell has a negative charge, as characterized by one or more methods known in the art (e.g., by measuring its zeta potential).

In some embodiments, the composite comprising a polyOH or polyCOOH shell has a negative charge of at least −10 mV. In some embodiments, the composite comprising a polyOH or polyCOOH shell has a negative charge of at least about −10 mV to −40 mV. In some embodiments, the composite comprising a polyOH or polyCOOH shell has a negative has a negative charge of at least about −15 mV to −30 mV. In some embodiments, the composite comprising a polyOH or polyCOOH shell has a negative charge of at least about −15 mV to −25 mV. In some embodiments, the composite comprising a polyOH or polyCOOH shell has a negative charge of at least about −20 mV to −25 mV. In some embodiments, the composite comprising a polyOH or polyCOOH shell has a negative charge of at least about −25 mV. In some embodiments, the composite comprising a polyOH or polyCOOH shell has a negative charge of at least about −20 mV.

In some embodiments, the composite comprising e.g., a shell comprising amine groups has a positive charge.

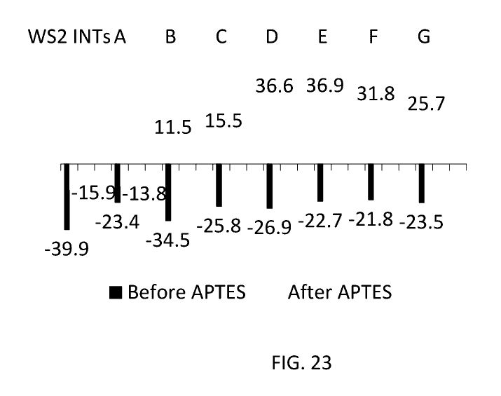

Reference is made to FIG. 23 presenting Zeta Potential values (mV) for e.g., untreated WS₂ NTs, WS₂ NTs having polyOH shell before and after APTES attachment thereto.

According to an aspect of some embodiments of the present invention, there is provided a nanostructure of metal chalcogenide (e.g., WS₂) having attached to at least one surface thereof chemically reactive nanoparticles. In some embodiments, the nanostructure is a nanotube. In some embodiments, the chemically reactive nanoparticles are magnetically responsive nanoparticles. In some embodiments, the chemically reactive nanoparticles are Fe₂O₃ nanoparticles. In some embodiments, the chemically reactive nanoparticles are fullerene nanoparticles. In some embodiments, the Fe₂O₃ nanoparticles are maghemite gamma (γ)-Fe₂O₃.

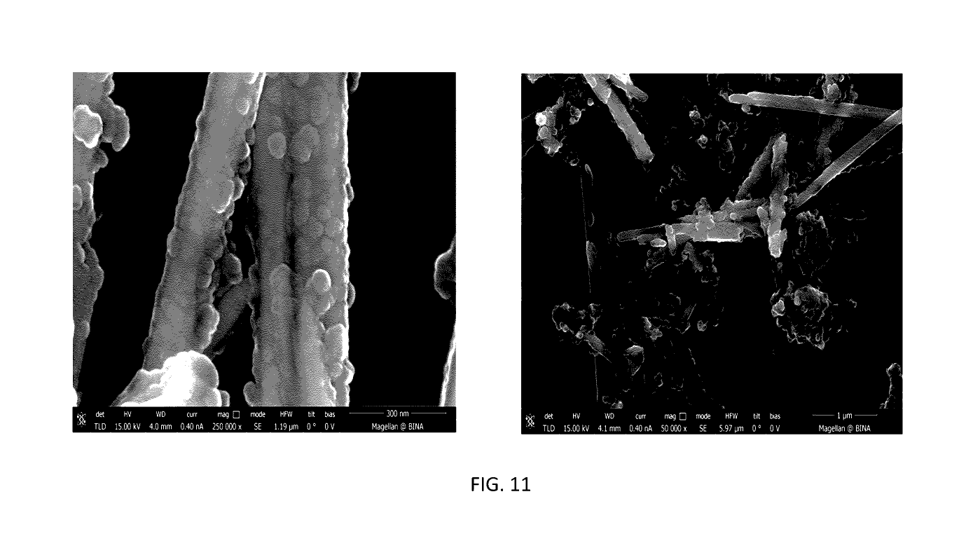

Reference is made to FIG. 11 which presents HRSEM images of synthesized maghemite (γ-Fe₂O₃) nanoparticles onto INT-WS₂.

In some embodiments, the nanostructure (e.g., nanotube) comprises cerium. In some embodiments, the nanostructure comprises a ligand or a complex.

In some embodiments, the nanostructure (e.g., WS₂) comprising a ligand or a complex is produced via an ultrasonic-assisted process.

In some embodiments, the Fe₂O₃ nanoparticles (NPs) are metal cation/complex-doped maghemite γ-Fe₂O₃([Lₙ]-γ-Fe₂O₃(Lₙ denotes a ligand). In some embodiments, Lₙ is a Lewis base ligand (e.g., an N/O/S-containing organic species/polymer). In some embodiments, the surface is modified via S atom affinity. In some embodiments, the ligand is derived from ceric ammonium nitrate, Ce^{IV}(NH₄)₂(NO₃)₆(CAN).

In some embodiments, the use of the term “ligand” is one ligand or a combination of two or more ligands. In some embodiments, the ligand is composed of at least two mixed ligands with or without any additional post-nanoparticle attachment chemical modification. In some embodiments, the ligand is a polymer. In some embodiments, the ligand is an organic molecule. In some embodiments, the ligand is polycationic. In some embodiments, the nanoparticle comprises a ligand that is mono or polydentate or that can be chemically modified by means such as, but not limited to, oxidation, when attached onto the nanoparticle surface. In some embodiments, the ligand is a small molecule. In some embodiments, the ligand is bound to the shell. In some embodiments, the core is devoid of a ligand. In some embodiments, the ligand is bound primarily to the shell.

In some embodiments, the ligand is a targeting ligand. In some embodiments, the targeting ligand is a molecule or a structure that provides targeting of the nanoparticle to a desired organ, a tissue, a cell receptor, or a cell. In some embodiments, the ligand includes, but without being limited thereto, proteins, peptides, antibodies, nucleic acids, sugar derivatives, or combinations thereof. In some embodiments, the nanoparticle further comprises targeting agents such that, when used as contrast agents, the particles can be targeted to specific diseased areas of the subject's body. In some embodiments, the nanoparticles may be used as blood pool agents. In some embodiments, the ligand is a biologically active molecule. In some embodiments, the ligand is a cytokine. In some embodiments, the ligand is a protein. In some embodiments, the ligand is a nucleic acid molecule such as, but not limited to, siRNA, RNAi, dsRNA, DNA, or any combination thereof. In some embodiments, the ligand is an organic molecule. In some embodiments, the ligand is a cell permeation molecule. In some embodiments, the ligand is an antibody. In some embodiments, the ligand has a biological activity such as, but not limited to, drug.

In some embodiments, the ligand is attached by way of covalent bonding, hydrogen bonding, adsorption, metallic bonding, Van der Waals forces, ionic bonding, or any combination thereof to the nanostructure. In some embodiments, the ligand is covalently bound to the cerium within the shell. In some embodiments, the ligand is coordinatively bound to the cerium within the shell.

In some embodiments, the ligand comprises a positively charged, negatively charged, or is a neutral organic moiety.

In some embodiments, the organic moiety comprises at least one Lewis basic heteroatom selected from, without being limited thereto, N, O, and S or any combination of N, O, and S.

In some embodiments, the ligand is a polymer. In some embodiments, the ligand is a Polyethylenimine (PEI). In some embodiments, the ligand is a Polyethylenimine r that is linear. In some embodiments, the ligand is a Polyethylenimine that is branched. In some embodiments, the ligand is a Polyethylenimine that has a MW of 2,000 to 80,000. In some embodiments, the ligand is a Polyethylenimine that has a MW of 4,000 to 25,000. In some embodiments, the ligand is a Polyethylenimine that has a MW of 25,000 to 40,000.

In some embodiments, the ligand is a polycationic polymer. In some embodiments, the ligand is a Polyethylenimine type cationic polymer and effective substitutes. In some embodiments, the ligand is a Polyethylenimine type cationic polymer having effective substitutes. In some embodiments, the ligand is Chitosan. In some embodiments, the ligand is Poly-L-lysine. In some embodiments, the ligand is Lysozyme. In some embodiments, the ligand is Diethylaminoethyl-dextran (DEAE-dextran). In some embodiments, the ligand is Polyornithine. In some embodiments, the ligand is Histone. In some embodiments, the ligand is Hexadimethrine bromide. In some embodiments, the ligand is Polyarginine. In some embodiments, the ligand is Protamine.

In some embodiments, the ligand is a marker. In some embodiments, the ligand is an imaging agent. In some embodiments, the ligand is a biomarker. In an additional embodiment the ligand is a radioactive isotope. In some embodiments, the ligand is a protein. In some embodiments, the ligand is a fluorophore. In some embodiments, the ligand is a cell-death facilitating agent.

In some embodiments, the Fe₂O₃ nanoparticles are positively charged super-paramagnetic maghemite (gamma-Fe₂O₃) nanoparticles that are doped (e.g., in a surface thereof) by controlled amounts of complexes/Ce^{3/4+} ([Ce^{3/4+}Lₙ]). In some embodiments, the term “cerium” includes or can be replaced with the term “cerium cations”. In some embodiments, the term “cerium” includes or can be replaced with the term “cationic cerium species”. In some embodiments, the term “cerium” includes or can be replaced with the term “cerium phase”. In some embodiments, the term “cerium” includes or can be replaced with the term “cerium (III/IV) cations”. In some embodiments, the term “cerium” includes or can be replaced with the term cerium (III). In some embodiments, the term “cerium” includes or can be replaced with the term cerium (IV). In some embodiments, the term “cerium” includes or can be replaced with the term “cerium phase (cerium (III/IV) cations)”.

In some embodiments, the nanotube is in the form of a core-shell. The term “core-shell” is described hereinabove. In some embodiments, the core comprises a metal chalcogenide. In some embodiments, the shell comprises [Lₙ]-γ-Fe₂O₃ nanoparticles, and optionally further comprises cerium ions. In some embodiments, the shell comprises [Lₙ]-Y—Fe₂O₃ nanoparticles, and further comprises cerium ions.

According to an aspect of some embodiments of the present invention, there is provided an article which comprises the disclosed hybrid composite. According to an aspect of some embodiments of the present invention, there is provided an article which comprises the metal chalcogenide nanotube comprising chemically reactive nanoparticles. Any article that may benefit from the disclosed composite and/or nanotube is contemplated.

In some embodiments, the article is for use in medicine. In some embodiments, the article for use in medicine is selected from all types and sizes of implants. In some embodiments, the article for use in medicine is a non-toxic biological implant. In some embodiments, the article for use in medicine is a bone tissue engineering system. In some embodiments, the article for use in medicine is a mobile functioning human joint. In some embodiments, the article for use in medicine is a tissue-penetrating device. In some embodiments, the article for use in medicine is a drug delivery hybrid system.

In some embodiments, the article is or comprises a mechanically reinforced polymeric 2D coating. In some embodiments, the article is or comprises a 3D matrix or fiber. In some embodiments, the article is or comprises 3D polymer printing structure. In some embodiments, the article is or comprises mechanically reinforced filtration membrane. In some embodiments, the article is or comprises a lubricant hybrid system. In some embodiments, the article is or comprises a conductive electrode component. In some embodiments, the conductive electrode component is a part in, without being limited thereto, a battery, a super-capacitor, a diagnostic device, a sensing device, and a field effect transistor (FET) sensor.

According to an aspect of some embodiments of the present invention, there is provided a process for producing the disclosed hybrid composite, comprising the steps of:

mixing bithiophene or a derivative thereof and a dehydrating agent; thereby synthesizing 2,2′-bithiophene-5-carboxaldehyde; and adding to the 2,2′-bithiophene-5-carboxaldehyde a nanostructure of metal chalcogenide or a metal dichalcogenide, thereby producing the hybrid material.

In some embodiments, the dehydrating agent is POCl₃. In some embodiments, the dehydrating agent is PCl₃. In some embodiments, the dehydrating agent is SOCl₂. In some embodiments, the dehydrating agent is SO₂Cl₂. In some embodiments, the dehydrating agent is SO₃. In some embodiments, the dehydrating agent is PCl₅. In some embodiments, the dehydrating agent is P₂O₅. In some embodiments, the dehydrating agent is VOCl₃. In some embodiments, the dehydrating agent is AlCl₃. In some embodiments, the dehydrating agent is TiCl₄. In some embodiments, the dehydrating agent is acetic anhydride. In some embodiments, the dehydrating agent is trifluoromethanesulfonic anhydride.

In some embodiments, the process allows combinatorial surface engineering. By “combinatorial surface engineering” it is meant to refer to a technique of differentially processing multiple regions of one or more surfaces, (e.g., in a coating or in a confomeric structure) as further described hereinthroughout.

According to an aspect of some embodiments of the present invention, there is provided a process for activation of INT-WS₂ by oxidation. In some embodiments, the process comprises a subsequent derivatization with APTES ((EtO)₃(Si(CH₂)₃NH₂).

In some embodiments, the WS₂—NTs are oxidized by an oxidizing agent (e.g., in the form of a solution) selected from, without being limited thereto, H₂O₂, HNO₃, K₂Cr₂O₇, K₂S₂O₈, NaClO, ceric ammonium nitrate (CAN), meta-chloroperoxybenzoic acid (MCPBA) and a Fenton reagent.

According to an aspect of some embodiments of the present invention, there is provided a synthesis of maghemite (γ-Fe₂O₃) NP onto inorganic nanotubes (INT) and/or inorganic fullerenes (IFs) nanomaterial.

In some embodiments, the process comprises a step of dispersion INT-WS₂ or INT-WS₂ in water.

In some embodiments, the process further comprises a step of bubbling an inert gas into the dispersion sample. In some embodiments, the process further comprises a step of ultrasonic irradiation of the dispersion. In some embodiments, the process further comprises a step of placed on a hotplate at e.g., 60° C., 65° C., 70° C., 75° C., 80° C., 85° C., 90° C., or 95° C., including any value therebetween. In some embodiments, the process further comprises a step of adding a solution comprising Fe ion (e.g., FeCl₂) and an acid (HCl) into the sample. In some embodiments, the process further comprises a step of adding NaNO₃ into the sample.

### DEFINITIONS

As used herein, the term “alkyl” describes an aliphatic hydrocarbon including straight chain and branched chain groups. Preferably, the alkyl group has 21 to 100 carbon atoms, and more preferably 21-50 carbon atoms. Whenever a numerical range; e.g., “21-100”, is stated herein, it implies that the group, in this case the alkyl group, may contain 21 carbon atom, 22 carbon atoms, 23 carbon atoms, etc., up to and including 100 carbon atoms. In the context of the present invention, a “long alkyl” is an alkyl having at least 20 carbon atoms in its main chain (the longest path of continuous covalently attached atoms). A short alkyl therefore has 20 or less main-chain carbons. The alkyl can be substituted or unsubstituted, as defined herein.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an —O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, alkoxy, nitro, amine, hydroxyl, thiol, thioalkoxy, thiohydroxy, carboxy, amide, aryl and aryloxy, depending on the substituted group and its position in the molecule. Additional substituents are also contemplated.

The term “halide”, “halogen” or “halo” describes fluorine, chlorine, bromine or iodine.

The term “haloalkyl” describes an alkyl group as defined herein, further substituted by one or more halide(s).

The term “haloalkoxy” describes an alkoxy group as defined herein, further substituted by one or more halide(s).

The term “hydroxyl” or “hydroxy” describes a —OH group.

The term “thiohydroxy” or “thiol” describes a —SH group.

The term “thioalkoxy” describes both an —S-alkyl group, and a —S-cycloalkyl group, as defined herein.

The term “thioaryloxy” describes both an —S-aryl and a —S-heteroaryl group, as defined herein.

The term “amine” describes a —NR′R″ group, with R′ and R″ as described herein.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine.

The term “heteroalicyclic” or “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino and the like.

The term “carboxy” or “carboxylate” describes a —C(═O)—OR′ group, where R′ is hydrogen, alkyl, cycloalkyl, alkenyl, aryl, heteroaryl (bonded through a ring carbon) or heteroalicyclic (bonded through a ring carbon) as defined herein.

The term “carbonyl” describes a —C(═O)—R′ group, where R′ is as defined hereinabove.

The above-terms also encompass thio-derivatives thereof (thiocarboxy and thiocarbonyl).

The term “thiocarbonyl” describes a —C(═S)—R′ group, where R′ is as defined hereinabove.

A “thiocarboxy” group describes a —C(═S)—OR′ group, where R′ is as defined herein.

A “sulfinyl” group describes an —S(═O)—R′ group, where R′ is as defined herein.

A “sulfonyl” or “sulfonate” group describes an —S(═O)₂—R′ group, where Rx is as defined herein.

A “carbamyl” or “carbamate” group describes an —OC(═O)—NR′R″ group, where R′ is as defined herein and R″ is as defined for R′.

A “nitro” group refers to a —NO₂ group.

A “cyano” or “nitrile” group refers to a —C—N group.

As used herein, the term “azide” refers to a —N₃ group.

The term “sulfonamide” refers to a —S(═O)₂—NR′R″ group, with R′ and R″ as defined herein.

The term “phosphonyl” or “phosphonate” describes an —O—P(═O)(OR′)₂ group, with R′ as defined hereinabove.

The term “phosphinyl” describes a —PR′R″ group, with R′ and R″ as defined hereinabove.

The term “alkaryl” describes an alkyl, as defined herein, which substituted by an aryl, as described herein. An exemplary alkaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

General

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find support in the following examples.

### EXAMPLES

### Example 1

### Polymerization of Conductive Polymers (CPs) onto INT-WS₂

In this study, a functionalization method for INT-WS₂ that uses readily available materials and equipment to produce an acidic, covalently bound shell of polyCOOH functional groups was performed.

In exemplary procedures, a new coating method of polythiophene derivatives using a similar Vilsmeier Haack type reaction by reacting 2, 2′-bitiophene-5-carboxaldehyde with INTs-WS₂ and POCl₃ as activating agent to react with outer layer surface S atoms of INT-WS₂ materials was performed. Scheme 1 summarizes the corresponding approach.

The 2^{nd} step polyTh-based oxidative liquid polymerization (LPP) was carried out using thiophene acetic acid (TAA) or 3,4-ethylenedioxythiophene (EDOT) as monomers and FeCl₃ as the oxidant agent (Scheme 2). This innovative method is very important since any conductive polymer can be polymerized onto the WS₂ surface leading to improved conductivity of composite WS₂. In addition, the WS₂ nanotubes can strongly mechanically reinforce deposited conductive polymers, which can improve the mechanical/functional stability of these polymeric composites in any desired device.

Next, Oxidative Polymerization of Conductive Polymers (CPs) onto WS₂ INTs—PolyThiophene-based conductive composite polyTh-WS₂ INTs was performed.

Herein, a suitable functionalization of starting INTWS₂ nanotubes has been performed via a method developed using the high electrophilic reactivity of a modified Vilsmeier-Haack (VH) reagent. This functionalization method produces polycarboxylated INT-WS₂ (polyCOOH f-INT-WS₂) that are readily dispersible in polar liquids including water. Thus, these polycarboxylation sites may serve as anchor points for secondary-step derivatizations, providing a versatile route for additional surface modifications through the well-known chemical reactivity of surface carboxylic acid groups (polyCOOH shell). These surface modifications enable matching interfacial compatibility towards both hydrophilic and hydrophobic composite materials based on a WS₂ inorganic core phase. In addition, subsequent functionalizations can include bi- or multifunctional species that enable covalent bonding with other materials/polymeric phases in specific compositions. This is a highly important feature for the controlled fabrication of hybrid nanocomposites that may contain both co-fillers and monomers, for example.

Covalently grown polythiophene (polyTh) polymers directly from tungsten disulfide inorganic nanotubes (INTs-WS₂) are described below, e.g., using thiophen-3-yl-acetic acid (TAA) monomer (schemes 2 and 3).

Without being bound by any particular theory, the concept of such a covalent growth combines two key steps as shown in scheme 4, and described hereinbelow. The first step is the covalently coupling/grafting of polycarboxylated INTs (polyCOOH-INTs) with a hydroxylated thiophene linker (thiophene-3-ethanol) affording nucleophilized thiophene-decorated INTs (Th-linker-INTs). This allowed anchoring of nucleophilic thiophene groups onto the surface of chemically modified polyCOOH-INTs in this 1st step, which acted as nucleation points for polyTh polymer growth and nanotube functionalization. The 2^{nd} step of this functionalization process is the in situ oxidative polymerization of functional mono-carboxylated TAA monomer (liquid phase polymerizations, -LPP conditions) in the presence of former Th-linker-INTs.

### Synthesis of 2,2′-bithiophene-5-carboxaldehyde

In exemplary procedures, 1.103 g of 2,2′ bithiophene (6.65 mmol) were dissolved in 10 ml of DMF. The solution was cooled to 4° C. in an ice bath and 650 μl of POCl₃ (6.93 mmol) were added drop-wise. The solution was stirred at 4° C. for 20 minutes and then heated to 60° C. and let stirred overnight. The solution was cooled to 4° C. and 10 ml of water was added in order to neutralize the excess of POCl₃. The water/DMF solution was added to a separation funnel and with 20 ml of ethyl acetate. The organic phase was extracted with ethyl acetate (20 ml×4 times) and recombined. Then, the organic phase was washed with water (20 ml×10 times) to get rid of the DMF. The organic phase was dried over MgSO₄ and filtrated to a 200 ml round bottomed flask and evaporated to obtain 1.231 g green solid (91.44% yield).

### Characterization

### H¹-NMR (acetone-d⁶) 300 MHz

δ=9.925 ppm, s, 1H. δ=7.92 ppm, d, (J=1.2 Hz, 5.1 Hz), 1H. δ=7.615 ppm, dd, (J=1.2 Hz, 3.6 Hz), 1H. δ=7.535 ppm, dd, (J=1.2 Hz, 3.6 Hz) 1H, δ=7.459 ppm, d, (J=3.9 Hz), 1H. δ=7.167 ppm, dd, (J=3.6 Hz, 5.1 Hz), 1H.

### C¹³-NMR (acetone-d⁶) 300 MHz

δ=183.7 ppm (C═O), δ=139.021 ppm (C—H), δ=129.5 ppm (C—H), δ=128.429 ppm (C—H), 6-127.281 ppm (C—H), δ=125.564 ppm (C—H).

### Synthesis of INTs-WS₂-2,2′ bithiophene

In exemplary procedures, 300 mg of 2,2′ bithiophene-5-carboxaldehyde (1.544 mmol) were dissolved in 30 ml THF and were cooled to 4° C. in an ice bath. Then, 400 μl of POCl₃ were added and stirred for 30 minutes. 350 mg of INTs WS₂ were added and the dispersion was refluxed overnight. The dispersion was cooled to 4° C. in an ice bath and 20 ml of water was added to neutralize the excess of POCl₃. The dispersion was poured into centrifuge tubes and separated in centrifuge (8000 rpm 5 minutes), and was next washed with ethanol and centrifuged 5 times (5 minutes 8000 rpm each rotation), and was next dried in vacuum to obtain 280 mg of INTs-WS₂-2,2′ bithiophene.

### Synthesis of INTs-WS₂-bithiophene-Polythiophene acetic acid

In exemplary procedures, 60 mg of INTs-WS₂-2,2′ bithiophene was dispersed in 5 ml of chloroform and then 120 mg of FeCl₃ (0.74 mmol) were added and stirred for 30 minutes at room temperature. Then, 120 mg of thiophene acetic acid (0.84 mmol) were added and the suspension was stirred for 1 hour in room temperature. The suspension was poured into Eppendorf and centrifuged (5 minutes, 8000 rpm). The INTs were washed with ethanol 5 times (5 minutes, 8000 rpm) and dried in vacuum to obtain 55 mg of INTs-WS₂-bithiophene-polythiophene acetic acid.

### Synthesis of INTs-WS₂-bithiophene-Polythiophene acetic acid/poly 3,4-ethylenedioxythiophene

In exemplary procedures, 60 mg of INTs-WS₂-2,2′ bithiophene were dispersed in 5 ml of chloroform and 120 mg of FeCl₃ (0.74 mmol) and stirred for 30 minutes at room temperature. Then, 30 mg/60 mg/90 mg of thiophene acetic acid (0.21 mmol/0.42 mmol/0.633 mmol and 90 mg/60 mg/30 mg of 3,4 ethylenedioxythiophene (0.633 mmol/0.42 mmol/0.21 mmol) respectively and stirred for 1 hour at room temperature. The suspension was poured in to Eppendorf and centrifuged (5 minutes, 8000 rpm). The INTs were washed with ethanol 5 times (5 minutes, 8000 rpm) and dried in vacuum to obtain 80-100 mg of INTs-WS₂-bithiophene-polythiophene acetic acid-poly-3,4-ethylenedioxythiophene.

### Synthesis of INTs-WS₂-bithiophene-poly-3,4-ethylenedioxythiophene

In exemplary procedures, 60 mg of INTs-WS₂-2,2′ bithiophene were dispersed in 5 ml of chloroform and 120 mg of FeCl₃ (0.74 mmol) and stirred for 30 minutes at room temperature. Then, 180 μl of 3,4 ethylenedioxythiophene (0.95 mmol) were added and stirred for 1 hour at room temperature. The suspension was poured into Eppendorf and centrifuged (5 minutes, 8000 rpm). The INTs were washed with ethanol 5 times (5 minutes, 8000 rpm) and dried in vacuum to obtain 80-100 mg of INTs-WS₂-bithiophene-poly-3,4-ethylenedioxythiophene.

### Coupling of 1,3 diaminopropane to INTs-WS₂— Polythiophene acetic acid/poly 3,4 ethylenedioxythiophene

In exemplary procedures, 30 mg of INTs-WS₂-polythiophene acetic acid/poly 3,4-ethylenedioxythiophene (25%, 50%, 75%) were dispersed in 3 ml of chloroform and 200 mg of 1-ethyl-3-(3-dimethylaminopropyl) carbodiimide hydro chloride (1.57 mmol) and stirred for 30 minutes. Then, 300 μl of 1,3 diaminopropane (3.6 mmol) and stirred overnight at room temperature. The suspension was poured into Eppendorf and centrifuged (5 minutes, 8000 rpm). The INTs were washed with ethanol 5 times (5 minutes, 8000 rpm) and dried in vacuum to obtain 25-30 mg of INTs.

### INTs-WS₂— Polythiophene acetic acid/poly 3,4 ethylenedioxythiophene cleavage

In exemplary procedures, 50 mg of INTs WS₂ polythiophene acetic acid/poly 3,4 ethylenedioxythiophene were dispersed in 3 ml dichloromethane. Then, 1 ml of TFA was added and stirred for 1 hour at room temperature. The INTs were separated in centrifuge and the supernatant was evaporated to obtain purple/black solid.

INTs-WS₂ and IFs-WS₂ NPs were functionalized with a shell of bithiophene followed by further shell of poly thiophene oxidative polymerization derivatives (EDOT, TAA and EDOT/TAA mix) using FeCl₃ as an oxidant agent. In order to quantify the amount of polymer coating on the WS₂ surface, several tests were applied, including TGA, Kaiser Test (after coupling 1, 3-diaminopropane to WS₂-bithiophene-PTAA) and quality tests such as FTIR, Raman, TEM (including diffraction), SEM, Element analysis and EPR.

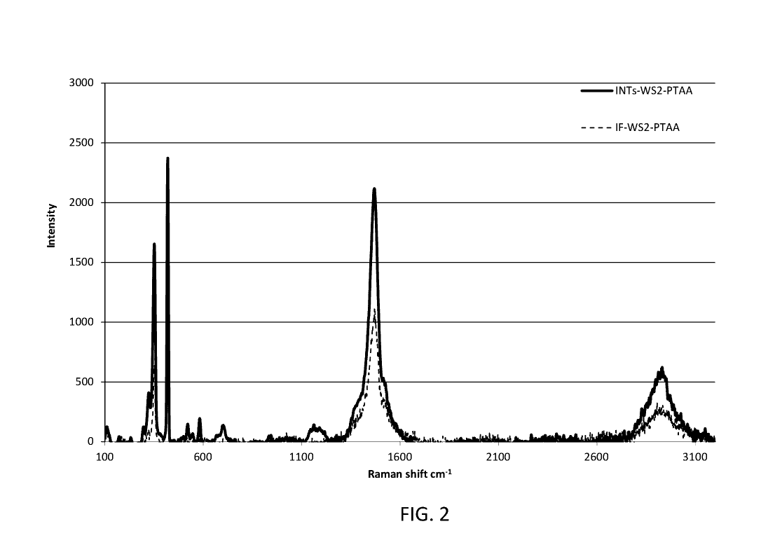

The Raman shifts showed the presence of polythiophene acetic acid (PTAA) on the INTs/IF-WS₂ surface. Room-temperature Raman spectra (FIG. 2) from a monolayer WS₂ region, using the 514 nm laser excitation, showed peaks at 2953 cm⁻¹ and 1473 cm¹ referring to C—H stretch and bend respectively. For λ_{exc}=514 nm, the Raman spectrum as dominated by the first-order modes: E¹_{2g}(Γ) at 349 cm⁻¹ and A_{1g}(Γ) at 418 cm⁻¹ which refers to S—W—S band.

In order to confirm the presence of carbon on the INTs/IF WS₂—PTAA/PEDOT, elemental analysis was carried out. The elemental analysis (Table 1, presenting Element analysis of INTs-WS₂—PTAA/PEDOT) showed 1.0917 percent of carbon in the INTs-WS₂-bithiophene before the polymerization. After the polymerization had been carried out, the amount of carbon was increased to 6%-30% which indicates the presence of polymer on the WS₂ surface.

TABLE 1

| | % | % | % | % | % |
|---|---|---|---|---|---|
| | Nitrogen | Carbon | Hydrogen | Sulfur | Oxygen |
| INTs-WS₂- | 0 | 1.0917 | 0.0912 | 29.6531 | 1.295 |
| bithiophene | | | | | |
| INTs-WS₂-PTAA | 2.2251 | 29.7946 | 2.2134 | 9.7206 | 13.491 |
| 1M/PEDOT 3M | | | | | |
| INTs-WS₂-PTAA | 0.9658 | 25.5475 | 1.7911 | 17.098 | 13.574 |
| 1M/PEDOT 1M | | | | | |
| INTs-WS₂-PTAA | 0.2893 | 11.0668 | 0.6248 | 23.0619 | 6.018 |
| 3M/PEDOT 1M | | | | | |
| INTs-WS₂-PTAA | 0 | 5.9991 | 0.4259 | 28.9686 | 4.236 |

The elemental analysis of IF-WS₂-bithiophene and IF-WS₂—PTAA/PEDOT (Table 2, presenting Element analysis of IF-WS₂—PTAA/PEDOT) gave similar results of organic quantity on the inorganic fullerenes surface as in the inorganic nanotubes.

TABLE 2

| | % | % | % | % | % |
|---|---|---|---|---|---|
| | Nitrogen | Carbon | Hydrogen | Sulfur | Oxygen |
| IFs-WS₂- | 0 | 0.7443 | 0.1159 | 23.0624 | 2.25 |
| bithiophene | | | | | |
| IFs-WS₂-PTAA | 0.0755 | 31.7185 | 2.4351 | 21.1552 | 17.522 |
| 1M/PEDOT 3M | | | | | |
| IFs-WS₂-PTAA | 0.5559 | 31.8598 | 2.5823 | 20.9082 | 16.675 |
| 1M/PEDOT 1M | | | | | |
| IFs-WS₂-PTAA | 0.1923 | 20.5940 | 1.4555 | 22.6140 | 12.73 |
| 3M/PEDOT 1M | | | | | |
| IFs-WS₂-PTAA | 0.1281 | 6.1169 | 0.4321 | 22.557 | 6.704 |
| IFs-WS₂-PEDOT | 2.1753 | 29.1617 | 2.8033 | 14.0539 | 15.984 |

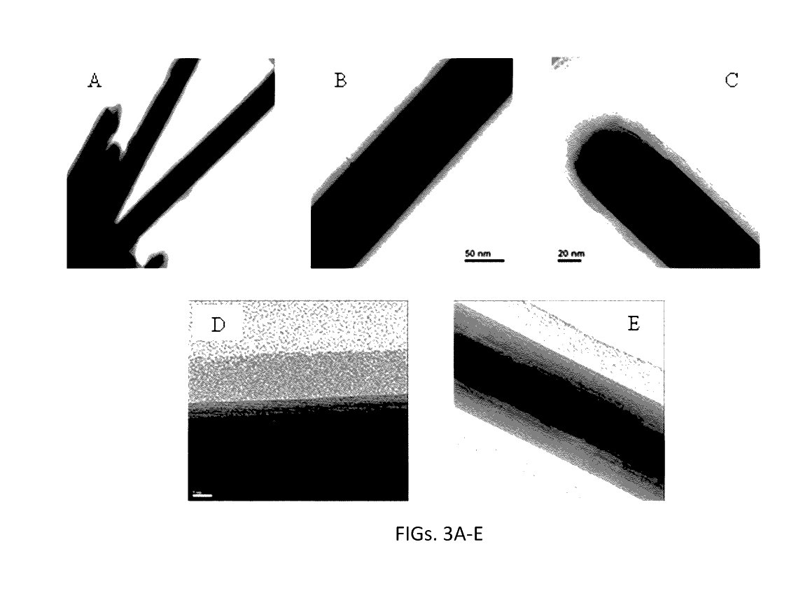

FIG. 3 presents HR-TEM (A-C) and Cryo-TEM analysis (D & E) of INTs-WS₂-PTAA Cryo-TEM and HR-TEM images (FIG. 3A-3E) show poly-thiophene acetic acid coating with a width around 11 nm.

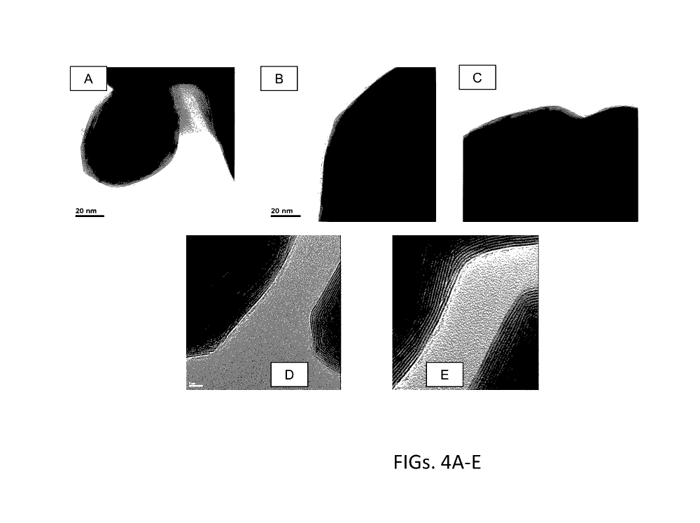

The TEM images of IFs-WS₂—PTAA (FIGS. 4A-4C) showed polymer coating on the inorganic fullerenes as well. FIGS. 3D and 3E were manipulated by HRTEM and show more clearly the polymer coating. The polymer coating width was narrower than the polymer coating on the inorganic nanotubes. This might be derived from the fact that the polythiophene was polymerized linearly while the fullerene had its special bended shape which hindered the polymer growth.

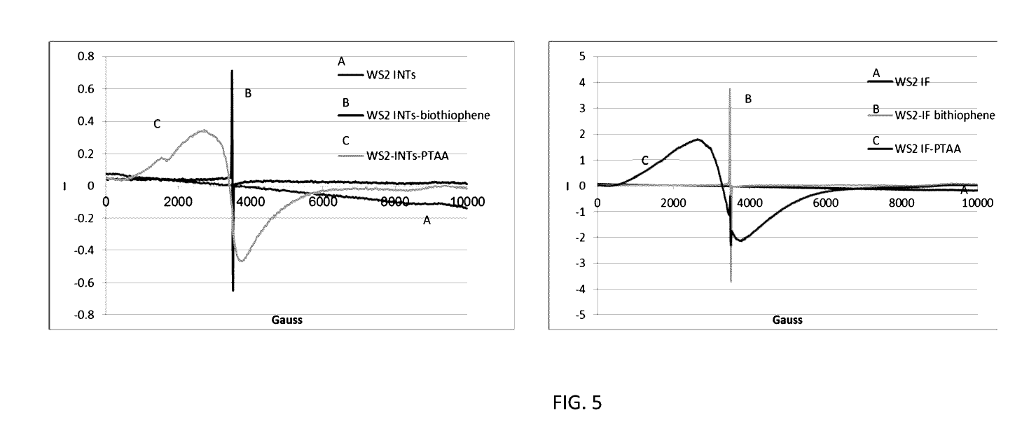

Electron paramagnetic resonance (EPR) analysis (FIG. 5) showed differences between WS₂-bothiophene and WS₂—PTAA. For WS₂-bithiophene, EPR showed a sharp peak which was derived from the sulfur's unpaired electrons. In the WS₂—PTAA spectrum on the other hand, a broad peak was observed which indicated that a polymer had been performed. The broad peak is derived from the polythiophene since it contains several numbers of radicals.

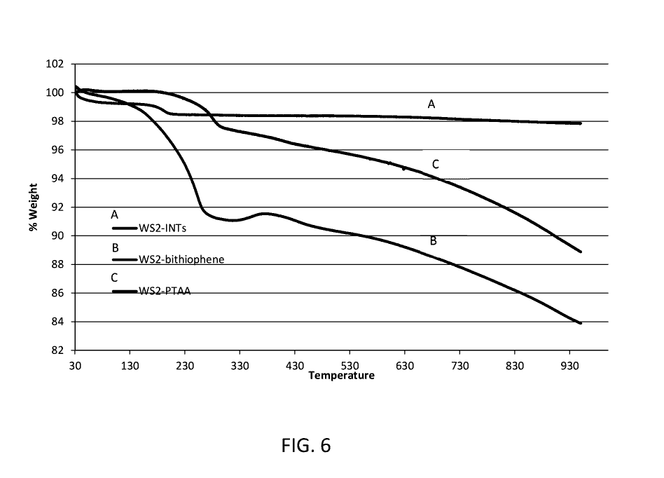

The TGA analysis (FIG. 6) showed significant weight reduction of organic material for WS₂-bithiophene and WS₂—PTAA (7.6% and 8.6%, respectively). The WS₂-INTs graph on other hand, showed reduction of 0.7% weight. These results indicates that organic material was attached to the INTs surface. Moreover, the stability of organic material was increased since even at 1000° C. the graph did not reach to plateau.

An additional exemplary procedure to covalently attach conductive molecules/polymers onto the WS₂ INT is described as follows:

Covalently growth of PTAA monomers onto the INTs surface:

following the polycarboxylation of the INTs, the growth of a thiophene polymer from the surface of the INTs involved two additional steps:

i. Covalent Coupling/Grafting of polyCOOH-INTs with Thiophene-Based Linkers

The preparation of competitive “nucleophilic” Th-containing CNTs were performed by activating polyCOOH-INTs with N′-(3-dimethylaminopropyl)-N-ethylcarbodiimide hydrochloride (EDC.HCl) [100 mg poly-COOH-INTs, 0.104 mmol EDC.HCl, 4 ml H₂O, 1 h, rt]. Then thiophene-3-ethanol was dissolved in CH₃CN and added to the suspension [0.104 mmol 2-(3-thienyl) ethanol, 1 ml CH₃CN, overnight, rt] to form an ester bond. The resulting product was washed with CH₃CN and distilled H₂O, decanted by centrifugation and dried under vacuum.

ii. In-Situ Oxidative Polymerization of PTAA Monomers onto Th-Decorated INTs (Th-Linker-INTs)

When thiophene linkers are covalently attached onto the INT, they readily interfere with the bulk oxidative Th-monomer polymerization causing polyTh-growth from the INT surface. The Th-linker functions act as nucleation a species for the polymerization of the desired polyTh polymer.

In exemplary procedures, the experimental conditions used in such an oxidative process were as follows: 1:1 weight Th-linker-INTs:Th-monomer, 1:5 M Th-monomer:oxidizing agent (FeCl₃) for a final concentration of 0.01 M CTAB surfactant. Each polymerization process included the dispersion of Th-linker-INTs (anhydrous CHCl₃) with the cationic surfactant cetyltrimethyl-ammonium bromide (CTAB) using an ultrasonicator bath (Bransonic, 42 kHz at full power) [50.0 mg Th-linker-INTs, 73.0 mg CTAB, 15 ml CHCl₃, 15 min] followed by the addition of the oxidant FeCl₃ [2.5 ml CHCl₃] followed by an additional 30 min sonication]. Th-monomers were previously dissolved in 2.5 ml CHCl₃ and added dropwise to the suspension [50.0 mg TAA, for polyThA]. The polymerization took place for 1 h under ultrasonication. The resulting polyThA-INTs composites were sequentially washed using MeOH and distilled H₂O before drying under vacuum (lyophilization).

HR-TEM microphotographs enabled the straightforward characterization of these composites, that is, the formation/precipitation of polyTh polymers around the INT basis, in a core-shell structure as shown in FIG. 7. TEM images show polythiophene acetic acid coating around the INTs with a thin nanometric width.

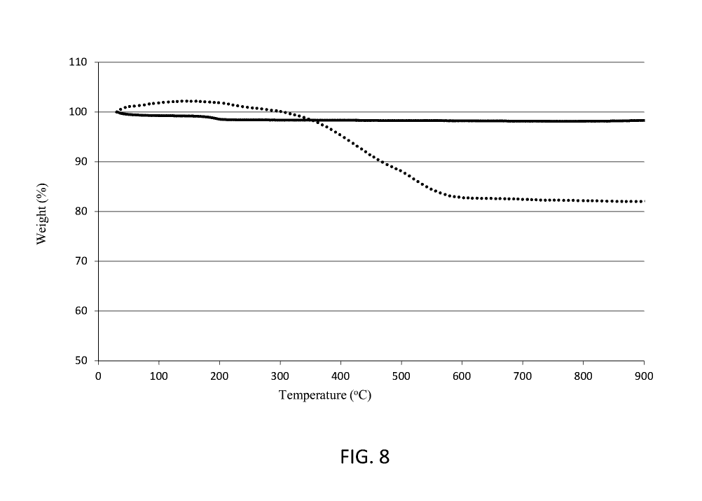

To evaluate the weight composition percent of polyTh-based polymer present on the INT surface, Ahermogravimetric Analyses (TGA) were performed for commercial starting INTs and polyThA INTs (FIG. 8).

The TGA analysis showed significant weight reduction of organic material for polyThA INTs (˜18%). The commercial starting INTs graph on other hand, showed reduction of 0.7% weight. These results indicates that organic material is attached to the INTs surface. These data also related to temperature window of 200-550° C. are relative. This might be rationalized by the expected stabilizing effect caused by the covalent grafting of polyThA polymeric chains onto corresponding INTs solid phase supports.

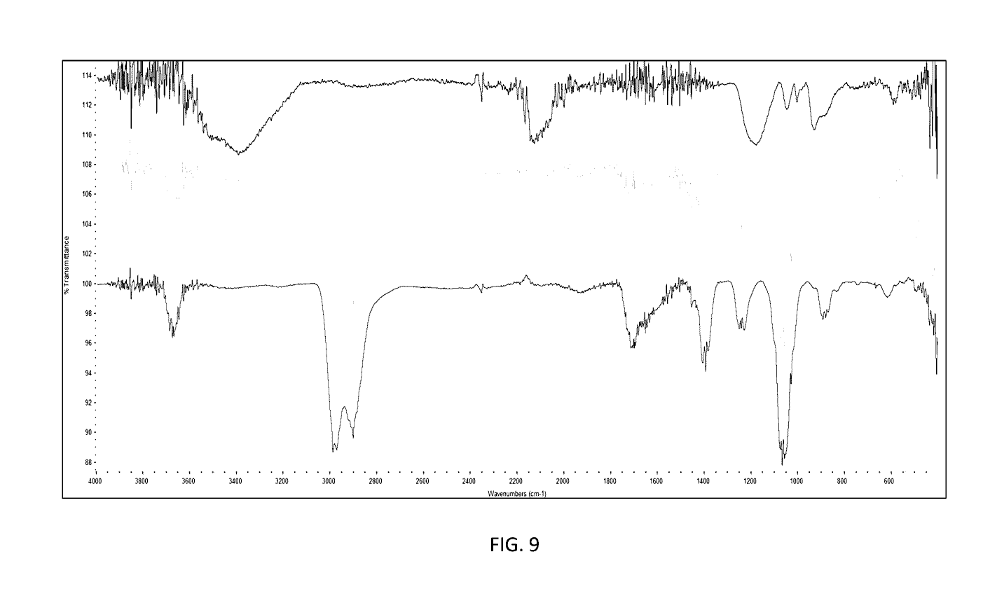

Preliminary FT-IR spectroscopic characterizations resulting CP/INT composite materials have been performed aiming at checking the presence of organic Polythiophene Acetic Acid phase. The analyzed polyThA INTs composites showed characteristic FT-IR peaks proving coherent chemical functionality. FT-IR spectra of commercial INTs, of polyCOOH-INTs, of Th-linker-INTs, of polyThA INTs composites are presented in FIG. 9.

Both 1600 and 1420 cm⁻¹ peaks correspond to symmetric and asymmetric thiophene ring stretching, respectively. These peaks can be observed in both Th-linker-INTs, of polyThA INTs composites. Moreover, the strong C—O stretching peak appearing at 1715 cm¹ and the broad O—H stretching peak at 2700-3600 cm⁻¹ are also characteristic of polyThA INTs composites.

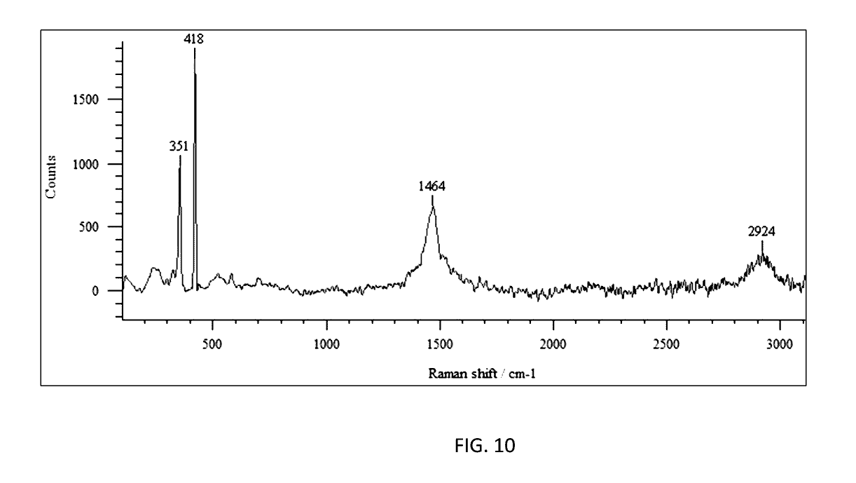

The Raman spectrum also showed the presence of polythiophene acetic acid (PTAA) on the INTs surface. Room-temperature Raman spectra (see FIG. 10) from a monolayer WS₂ region, using the 514 nm laser excitation, showing peaks at 2924 cm⁻¹ and 1464 cm⁻¹ refer to C—H stretch and bend respectively.

For λ_{exc}=514 nm, the Raman spectrum is dominated by the first-order modes: E¹_{2g}(Γ) at 349 cm⁻¹ and A_{1g}(Γ) at 418 cm⁻¹ which refers to S—W—S band.

The nanocomposite surface charge was evaluated by ζ (zeta) potential measurements after each modification (Table 3 below). Preliminary studies were performed to characterize the surface charge of INTs in dispersion state. Commercially available INTs were found to be negatively charged, having a ζ potential of −27.6 mV. After the polycarboxylation process, the ζ potential of polyCOOH-INTs became slightly more negative, that is, reaching a value of −27.9 mV. While the average ζ potential of the Th-linker-INTs composites was −14.3 mV and polyThA-INT composites was −21.7 mV.

In order to confirm the presence of carbon on the polyThA-INT composites, element analysis was carried out. Elemental analysis of commercial INTs, of polyCOOH-INTs, of Th-linker-INTs, of polyThA INTs composites afforded atomic percentage values of C, O, S, N, and H elements for each sample (as presented in Table 3).

TABLE 3

| | Zeta potential (mV) |
|---|---|
| INTs | −27.6 |
| Polycarboxylated-INTs | −27.9 |
| Th monomer modified INTS | −14.3 |
| Polymerization of PTAA monomers | −21.7 |
| onto Th-decorated INTs | |

The high amount of the polymeric phase in polyThA INTs was confirmed by elemental analysis that showed a high amount of C, O and H elements in polyThA INTs. Table 4 presents an elemental analysis of commercial INTs, of polyCOOH-INTs, of Th-linker-INTs, of polyThA INTs composites.

TABLE 4

| | C | O | S | N | H |
|---|---|---|---|---|---|
| Commercial INTs | 0 | 0.634 | 22.5281 | 0 | 0 |
| PolyCOOH-INTs | 0.403 | 0.58 | 19.2445 | 0.0851 | 0.0296 |
| Th-linker-INTs | 1.193 | 0.511 | 25.0128 | 0.0991 | 0.1177 |
| PolyThA INTs | 9.9025 | 3.318 | 22.5809 | 0.192 | 0.6766 |

To quantify the outer COOH groups present on the surface of both polycarboxylated INTs and polyTh-based INT composites, a highly sensitive UV spectroscopic Kaiser test was used. The products contained respectively 0.1 and 0.20 mmol NH₂ groups/g, as determined by the Kaiser test.

### Example 2

### Growth and Attachment of Maghemite (γ-Fe₂O₃) Nanoparticles onto WS₂ INT

Herein, a magnetically responsive INT-WS₂ via S affinity for metallic cations (Fe^{2/3+}) by a controlled growth of maghemite (γ-Fe₂O₃) nanoparticles onto inorganic INT-WS₂ is presented.

This part is aimed at investigating the feasibility to fabricate/grow magnetically responsive maghemite (γ-Fe₂O₃) nanoparticles onto tungsten disulfide nanotubes.

### Synthesis of Maghemite (γ-Fe₂O₃) NP onto INT and IF Nanomaterials

In exemplary procedures, 50 mg of WS₂(s) Inorganic Nanotubes (INTs) or WS₂(s) Inorganic Fullerenes (IFs) and 5.00 mL of deionized water were placed in a vial. Argon (g) was then bubbled into both vials. The samples were then placed in an ultrasonic bath for 8 minutes. They were then placed on a hotplate at 85° C., each with a magnetic stirrer. 0.8 g FeCl₂ were dissolved in 5 ml 3.2% HCl and were added to the sample vials followed by an addition of 150 mg NaNO₃ that was dissolved in 5 ml H₂O. Then the pH of the solution was raised to 10.5 with 1M NaOH (aq). INT-mag were isolated from the supernatant by placing the reaction vial close to an external magnet (0.5 T) so the coated INT were attached to the magnet, and the supernatant was changed five times. The resulting sediment of each vial was freeze-dried overnight in a lyophilized.

INT-mag nanoparticles were isolated from the supernatant by placing the reaction vial close to an external magnet (0.5 T).

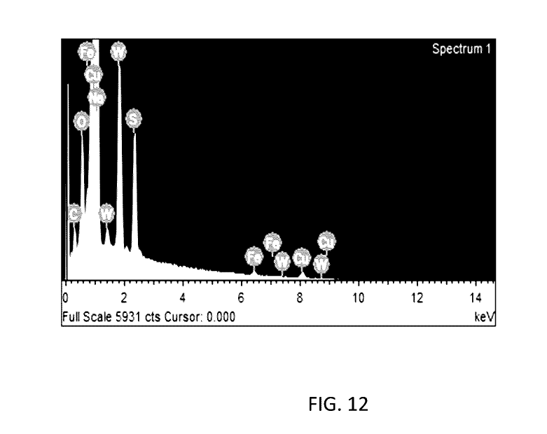

The maghemite nanoparticles polymerized and covered the entire surface of the WS₂ INT. FIG. 11 presents HRSEM images of synthesis of maghemite (γ-Fe₂O₃) nanoparticles onto WS₂ INT. FIG. 12 presents elemental analysis as done by HRSEM-EDX. The NPs size was approximately 65 nm. Table 6 below presents variable concentrations of best oxidizing agent solutions.

TABLE 6

| Element | Weight % | Atomic % |
|---|---|---|
| C K | 27.46 | 65.13 |
| O K | 3.85 | 6.85 |
| S K | 6.28 | 5.58 |
| Fe K | 0.47 | 0.24 |
| W M | 20.89 | 3.24 |

The elemental content of the tungsten di-sulfide INT cover with maghemite nanoparticles was measured by EDX. High content of tungsten and sulfur originated in the INT and Fe from the maghemite nanoparticles was noticed.

### Example 3

### Activation of WS₂—NTs by Oxidation and Subsequent Derivatization with 3-Aminopropyltriethoxy Silane (APTES)

Controlled Oxidation of WS₂ NTs:

In exemplary procedures, 150 mg of WS₂—NTs were placed into a glass bottle and 15 ml of [0.02-0.4 M] selected oxidizing solution [such as H₂O₂, HNO₃, K₂Cr₂O₇, K₂S₂O₈, NaClO, ceric ammonium nitrate (CAN), meta-Chloroperoxybenzoic acid (MCPBA) and Fenton reagent] was added. The bottle capped and sonicated in sonicator bath for 30 min.

The mixture was washed 5 times with bi-distilled water (to neutrality) (centrifugation 11,000 RPM, 10 min, 4° C.), and then likewise washed three times with THF. Between each centrifugation step, the solid product was dispersed in water/THF using 4 min of sonication. The product was vacuum dried at 40° C., overnight and stored in capped vials at room ambient.

One of the many oxidation species that are accepted to be on the surface of oxidized WS₂ NTs is hydroxyl groups. These hydroxyl groups allow further step of surface modification with silane groups, such as 3-aminopropyltriethoxy silane (APTES), 3-(Ethoxydimethylsilyl)propylamine, 3-(Mercaptopropyl)triethoxysilane etc.

Table 7 presents variable concentrations of best oxidizing agent solutions.

TABLE 7

| Oxidizing agent | Con [M] | Solvent |
|---|---|---|
| H₂O₂ | 0.3 | water |
| K₂Cr₂O₇ | 0.4 | water |
| CAN | 0.04 | water |
| MCPBA | 0.02 | DMF |
| Fenton | 0.02 | water |
| (1:1 eq of H₂O₂:FeSO₄•7H₂O) | | |

### Example 4

### Derivatization with 3-Aminopropyltriethoxy Silane (APTES)—Functionality Quantification

In exemplary procedures, the resulted oxidized WS₂—NTs (80 mg) were dispersed for five min in 80 ml of THF, using bath sonicator. Then, 1.5 ml of APTES (3.42 mmol) were added to the dispersion and stirred for 20 h at 60° C. After cooling to room temperature, the mixture was centrifuged (11,000 RPM, 10 min, 4° C.) and the supernatant discarded. The solid product was re-dispersed in THF (4 min of sonication) and likewise centrifuge for a total of five washings. The product was vacuum dried at 40° C., overnight, and stored in capped vials at room ambient.

Table 8 below presents elemental analysis for untreated-WS₂-INTs, Ox-WS₂-INTs (treatment with H₂O₂, K₂Cr₂O₇, CAN, MCPBA, and Fenton), and APTES-WS₂-INTs (additional treatment of APTES to these Ox-WS₂-INTs) in weight percentage.

TABLE 8

| Treatment | C % | H % | O % | N % | S % |
|---|---|---|---|---|---|
| untreated | 1.42 | 0.28 | 0.78 | 0.52 | 23.28 |
| H₂O₂ | 0.46 | 0.02 | 0.43 | 0.00 | 26.21 |
| APTES-H₂O₂ | 9.55 | 1.87 | 6.50 | 3.33 | 17.07 |
| K₂Cr₂O₇ | 1.55 | 0.26 | 2.06 | 0.00 | 23.06 |
| APTES-K₂Cr₂O₇ | 6.62 | 1.54 | 4.11 | 1.93 | 13.81 |
| CAN | 0.44 | 0.20 | 1.22 | 0.00 | 18.88 |
| APTES-CAN | 2.57 | 0.62 | 3.01 | 0.70 | 18.88 |
| MCPBA | 0.16 | 0.00 | 0.13 | 0.00 | 23.74 |
| APTES-MCPBA | 6.83 | 1.10 | 1.75 | 2.09 | 18.25 |
| Fenton | 0.49 | 0.13 | 1.65 | 0.14 | 20.29 |
| APTES-Fenton | 9.65 | 1.61 | 3.89 | 2.73 | 15.35 |

According to the N % in elemental analysis results, it can be seen that different oxidizing agents led to different degree of APTES attachment. The N % after the APTES derivatization of H₂O₂, K₂Cr₂O₇, CAN, MCPBA, and Fenton-WS₂-INTs was 3.33, 1.93, 0.70, 2.09, and 2.73, respectively.

TEM images showed that the oxidation step had created minor damages to the surface of the NTs and had caused exfoliation in some areas (FIG. 13 A-D). In addition, after the APTES attachment an organic coating around the tubes was observed, as a result of APTES polymerization (FIG. 13 E-F).

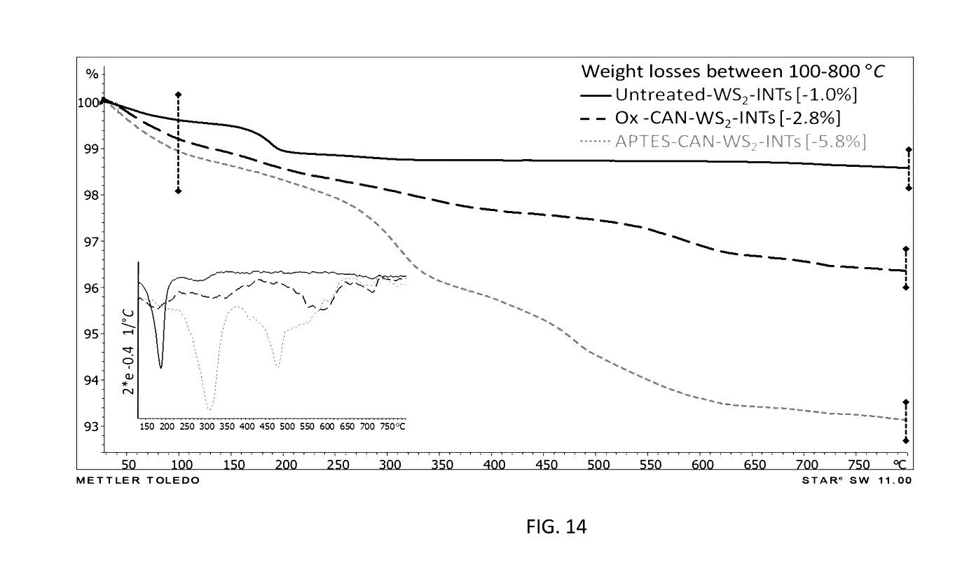

To evaluate the additional modification weight of oxidation and APTES attachment on the WS₂-INTs surface, Thermogravimetric Analyses (TGA) were performed for all the materials synthesized over all stages, including untreated-WS₂-INTs, Ox-WS₂-INTs, and APTES-WS₂-INTs. An illustrative example after the treatment with CAN reagent is shown in FIG. 14. TGA measurements revealed a 1.0% weight loss for untreated-WS₂-INTs, which likely resulted from INTs impurities or/and thermally unstable tubes. Weight losses of 2.8% and 5.8% to Ox-CAN-WS₂-INTs and APTES-CAN-WS₂-INTs respectively, and corresponded to the tube exfoliation and/or additional organic phase.

Moreover, the DTG (derivative thermogravimetry) plots of these three samples (inset 2), shows an interesting phenomenon—the peak at ˜200° C. of the untreated-WS₂-INTs which corresponds to the organic impurities of the INTs is almost disappeared after the oxidation step. The peaks at ˜600° C. and ˜700° C. of the Ox-CAN-WS₂-INTs are likely correspond to the tubes that damaged by the oxidation process. Regarding the APTES-CAN-WS₂-INTs sample, the peaks at ˜300° C. and ˜470° C. are related to the organic phase of the APTES addition.

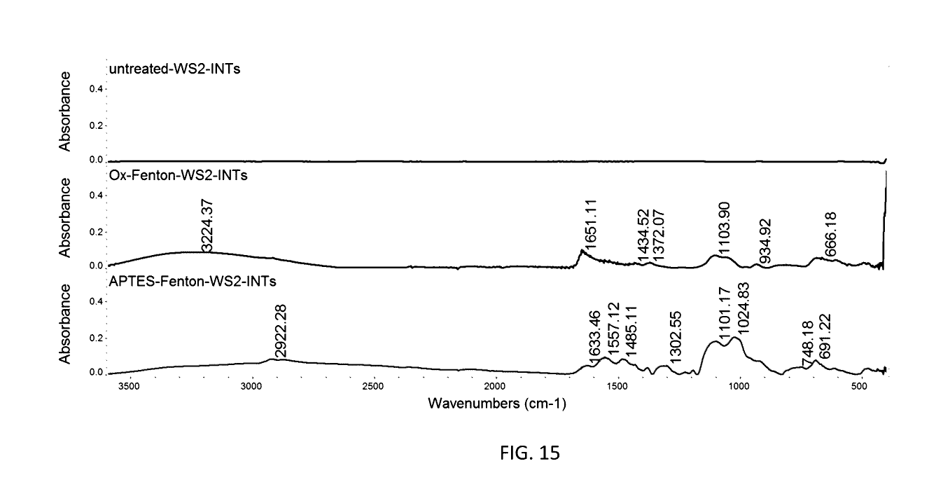

FIG. 15 is an illustrative example, showing the differences in the FTIR spectra after each step of modification, when the Fenton reagent is used. It can be seen that after the treatment with Fenton reagent (Ox-Fenton-WS₂-INTs spectra), a broad peak appears at ˜3200 cm⁻¹ which corresponds to the O—H stretch. After the attachment of APTES additional peaks appear such as the C—H stretch at ˜2922 cm⁻¹, N—H bend between 1550-1640 cm⁻¹, —CH₂— bend at ˜1485 cm⁻¹, C—N stretch at ˜1300 cm⁻¹, Si—O stretch at ˜1100 and 1024 cm⁻¹, and Si—C stretch at ˜748 cm⁻¹.

### Example 4

### Preparation of Polycarbohydrate-Coated WS₂ Nanotubes

In this Example an S-Glycosidation of inorganic INT-WS₂ nanotubes and nanoparticles towards functional composite polysaccharide/INT-WS₂ nanomaterials & subsequent surface derivatizations (Silicate Chemistry) is presented. That is, a new method for functionalizing INT-WS₂ nanotubes/nanoparticles with a polycarbohydrate-like shell containing free hydroxyl surface groups is presented (Scheme 5).

The method is based on a unique Lewis acid-activated thioglycosylation reaction between the nanotube surface and one chosen carbohydrate unit (see scheme 5 for details). It is shown that polysaccharide shell thicknesses may be readily controlled by adjusting sugar concentrations (see experimental parts below). Then and in a 2^{nd} step, a further reaction between such resulting polyOH-functionalized nanotubes and APTES (amine-terminated silicate) enabled quantification/tracking for optimization of polyOH surface groups concentrations as anew step towards further different surface functionalities (silicate chemistry). This pathway may help to improve existing applications of TMDs nanostructures and further expand their range of applications.

In addition, further polyOH shell may be exploited for derivatization of such intermediate core-shell composites with bifunctional silicates that contain for example a terminal amine (NH₂) group. The reaction with functional silicates is a second-level functionalization, which may improve the corresponding incorporation of resulting f-WS₂ ITs in various polymers.

In exemplary procedures, the first step in the formation of polycarbohydrate-coated WS₂ nanotubes is the attachment of saccharide units to the surface of the nanotubes. This step is based on a synthetic concept of thioglycosylation by Lewis acid-activation of the anomeric carbon of the saccharide unit. The Lewis acid boron trifluoride etherate (BF₃.Et₂O) was used both for acetylation of the unprotected sugars and for anomeric activation to thioglycosylation.

WS₂ nanotubes, due to their exterior sulfur atom layer, may be used as nucleophiles instead of thiols. Instead of starting from an unprotected sugar, a pentaacetate-protected sugar was used. For the preparation of saccharide-coated nanotubes, glucose pentaaceate and WS₂ nanotubes in different sugar/sulfur molar ratios (1, 2, 3, 4, 5, 8-marked A, B, C, D, E, F, G, respectively) were mixed in chloroform. The Lewis acid BF₃.Et₂O was then added in a constant molar ratio of 2.65 acid/sugar. The reactions took place in 48 mL pressure tubes at 60° C. (see below detailed procedures).

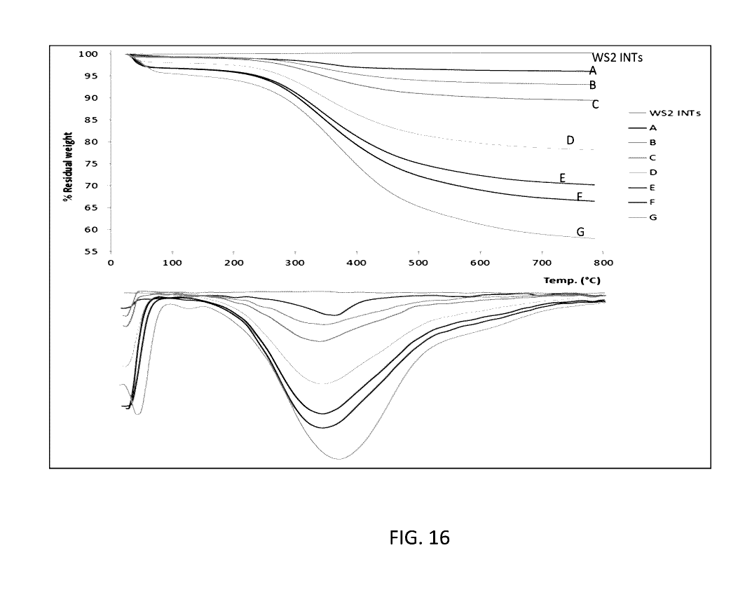

To evaluate the weight percentage of organic matter out of the coated nanotubes, a thermogravimetric analysis (TGA) and derivative thermogravimetric analysis (DTG) was used. WS₂ oxidizes to WO₃ when heated in presence of oxygen, therefore TGA analysis was conducted under nitrogen. The curves in FIG. 16 show a weight loss over a wide temperature range (typical for heating under nitrogen, compared sharper ranges for combustion). From the DTG curves, weight loss is shown in the range of 150−792° C. Table 9 which presents TGA weight losses of samples A-G and untreated WS2 NTs shows that the main weight loss, assigned to the organic matter in the composite, increases with the sugar molar ratio.

TABLE 9

| | | % Wt. |
|---|---|---|
| | Sugar/sulfur | loss |
| | molar ratio | 150-792° C. |
| WS₂ NTs | 0 | 0.2 |
| A | 0.5 | 3.2 |
| B | 1 | 6.4 |
| C | 2 | 9.7 |
| D | 3 | 19.7 |
| E | 4 | 26.4 |
| F | 5 | 30.0 |
| G | 8 | 37.0 |

Transmission electron microscopy (TEM) images (FIG. 17) enable the correlation of the molar ratio of the saccharide unit used in the reaction with the amount of the organic matter present in the composite, for obtaining the range of coating thicknesses (from few nanometers up to tenths of microns). In addition, the coating seems to be fully conformal with the exact contour of the nanotube. This suggests a specific surface chemistry between the polycarbohydrate layer and the outer wall of the nanotube. Based on these observations of increasing coating thicknesses, it is assumed, without being bound by any particular theory, that after the thioglycosylation reaction between the saccharide unit and the nanotube walls, a Lewis-acid-driven polymerization step follows. This polymerization may be of saccharide units or of their decomposition products. No electron diffraction pattern was obtained from the coatings, suggesting an amorphous structure of the polycarbohydrate shell.

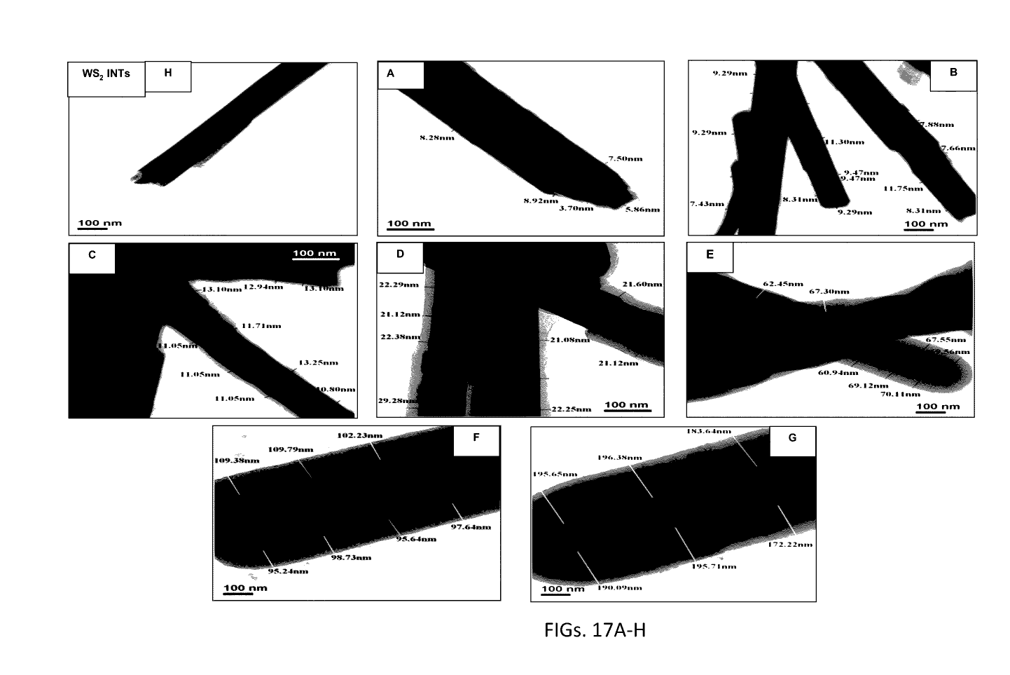

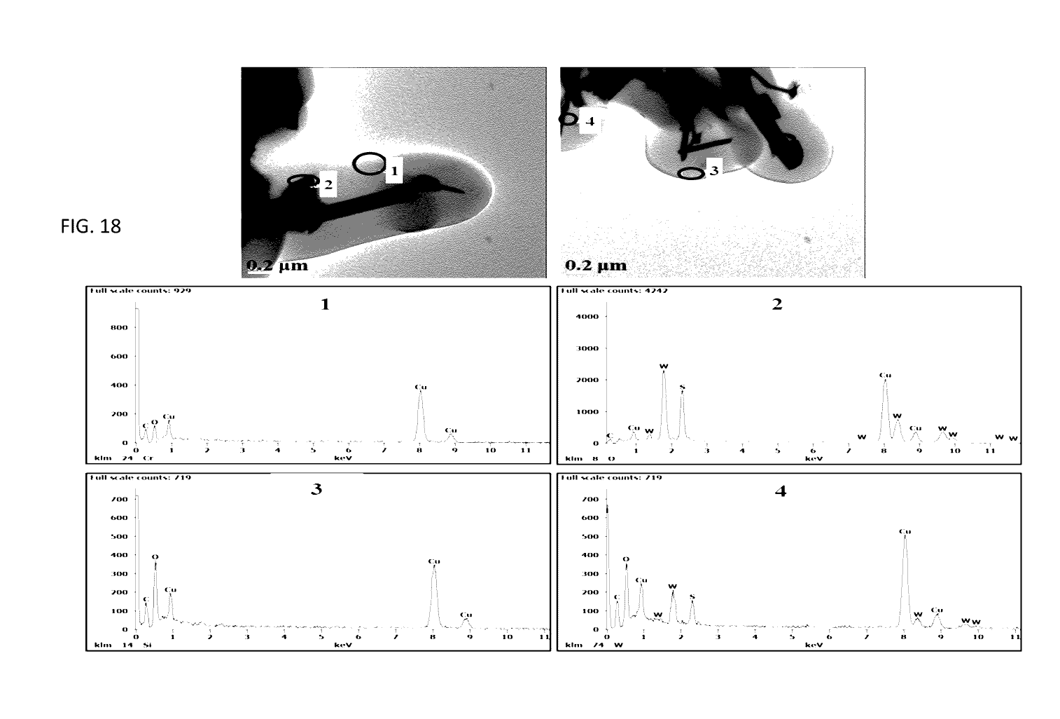

Energy-dispersive spectroscopy (EDS) spectra taken from four different points in sample G (FIG. 18) show the clearly defined distribution of elements in the sample. The spectra, taken from areas 1 and 3, which include only the polycarbohydrate coating, show the presence of carbon and oxygen, and the absence of tungsten and sulfur. In the spectrum taken from area 2, the tungsten and sulfur peaks are dominant compared to the weak peaks of carbon and oxygen, as one would expect looking on the scanned area. In the spectrum taken from area 4, all four elements are noticeably present. The origin of copper peaks is the grids on which the samples were prepared.

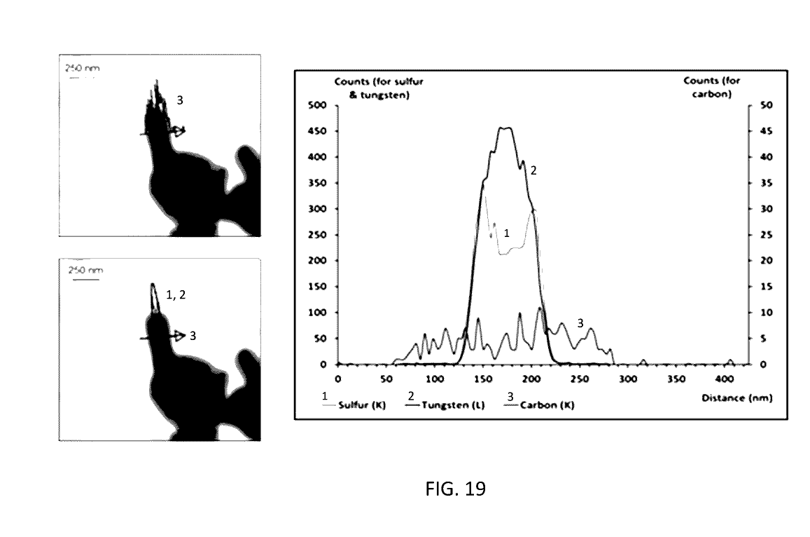

Scanning transmission electron microscopy (STEM) and energy-dispersive spectroscopy (EDS) line scan were done to confirm the core-shell morphology of the coated nanotubes. Elemental carbon (red curve, FIG. 19) is presented on a secondary axis because carbon, a light element, has a much lower signal compared to the heavier sulfur and tungsten. The profile plot clearly shows the presence of a 3D carbon-based coating around a tubular core containing tungsten and sulfur.

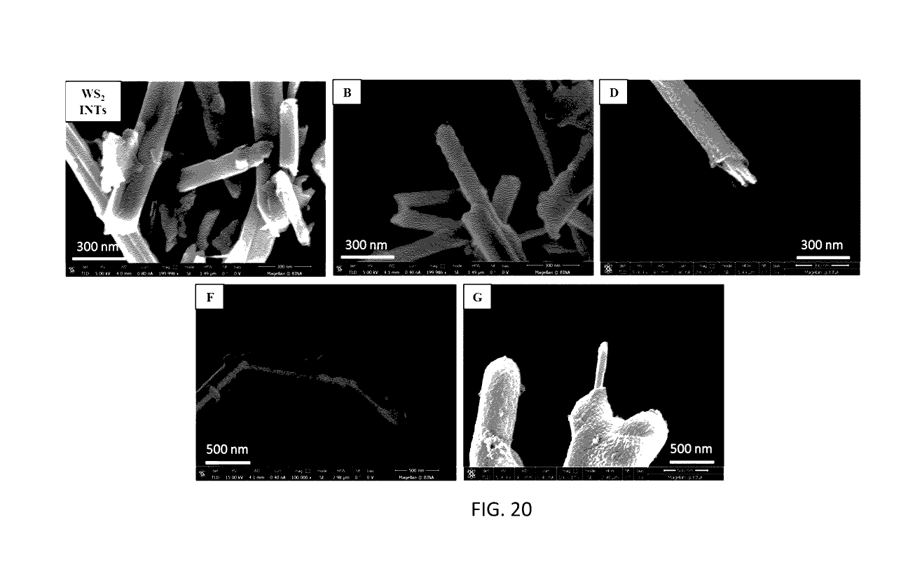

High-resolution scanning electron microscopy (HRSEM) images were taken to clearly present the conformal 3-dimentional structural coating around nanotubes. Samples B, D, F and G, compared to bare nanotubes, show different thicknesses of coatings (FIG. 20).

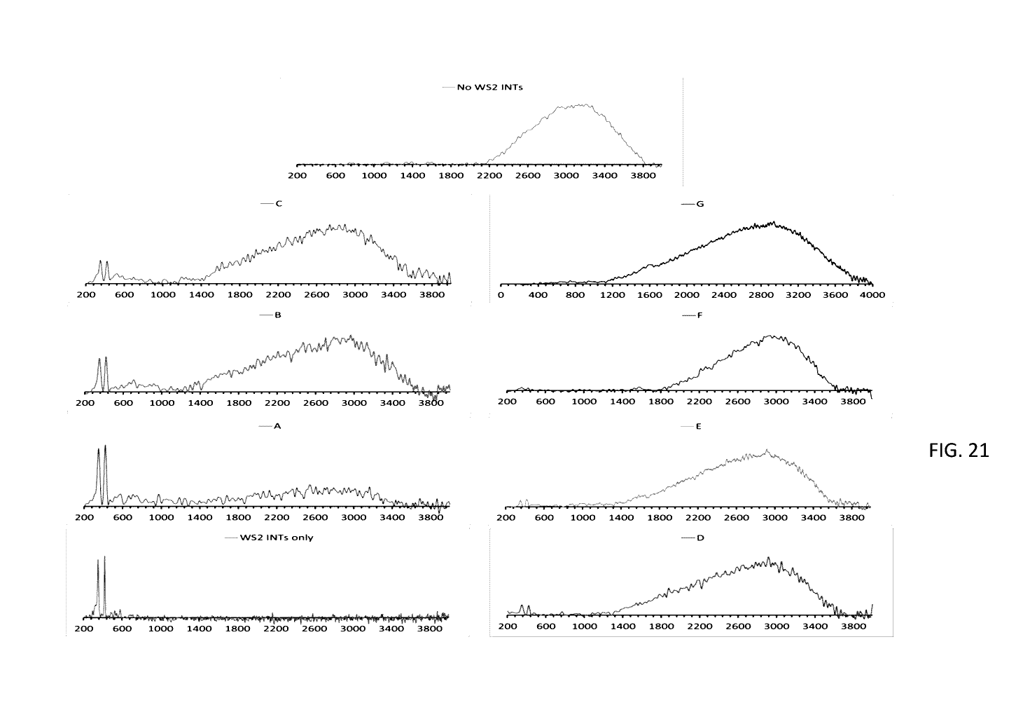

Raman spectra (FIG. 21), acquired with a 514 nm laser, show the presence of two sharp peaks—at 351 cm⁻¹ and 417 cm⁻¹, assigned to S—W—S stretches appearing in all spectra. Also present is a broad peak with a maximum at approximately 3,000 cm⁻¹ assigned to aliphatic C—H stretches. This peak is broad most likely because the organic coating is amorphous. Intensity ratio between this peak and the former two increases with the coating thickness.

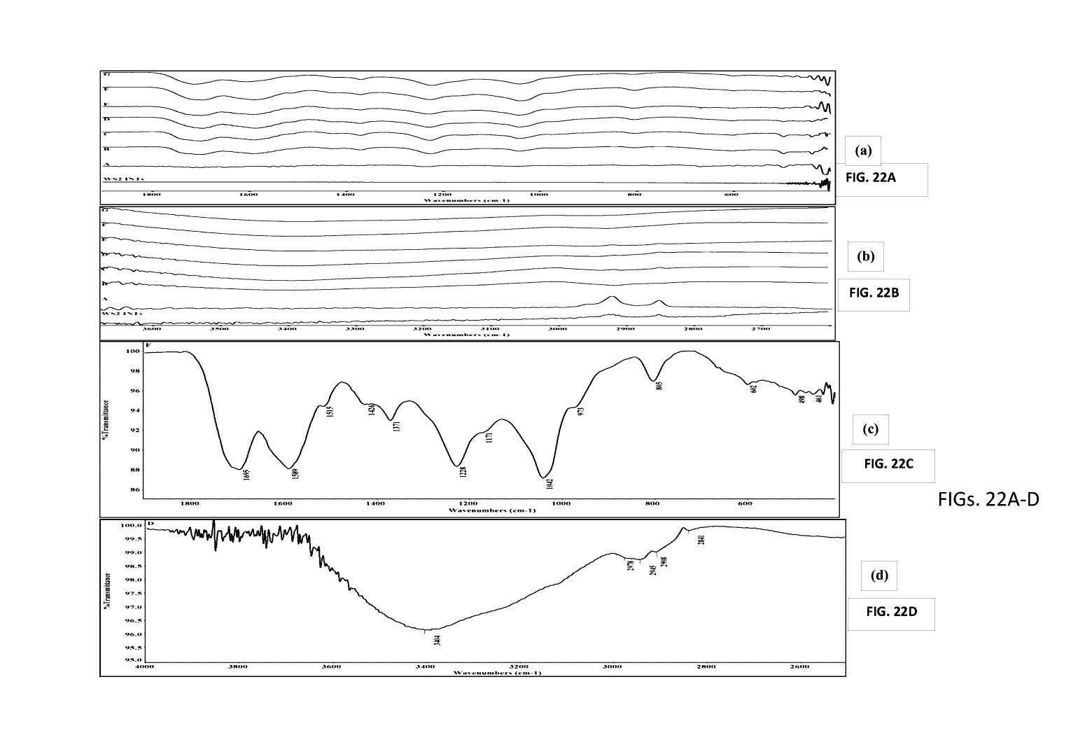

ATR-FTIR transmittance spectra of samples A-G (FIG. 22) show a very similar picture for all coating thicknesses, starting from sample B (plots a and b). As seen in the electron microscopy images, sample A is only practically coated. Plots c and d are focused on sample F, and show more conveniently the positions of the bands. Along with the color change observations during the coating process, these band positions may suggest, without being bound by a particular mechanism, the formation and polymerization of HMF (5-hydroxymethylfuraldehyde), a well-known degradation product of hexoses, that could have been formed under the reaction conditions—heat and an acidic medium. The bands at 805, 973 and 1042 cm⁻¹ may be assigned to C—H out-of-plane bending in furan unit. The bands at 1171 and 1228 cm⁻¹ may be assigned to rocking vibrations of the hydrogen atoms on the furan unit. The bands at 1371, 1426, 1515 and 1589 cm⁻¹ may be assigned to carbon-carbon double bond stretching on the furan unit. The band at 1695 cm⁻¹ may be assigned to the C═O stretching of the aldehyde moiety. The bands between 2900-3000 may be assigned to C—H stretching, and the broad band around 3400 cm⁻¹ is typical for the stretching of the hydroxyl moiety.

### Example 5

### Conjugation of Coated Functional Nanotubes with APTES

As above-mentioned, the polycarbohydrate shell that coats the chemically modified WS₂ nanotubes is most probably amorphous. Nonetheless, it is assumed, without being bound by any particular mechanism, that the resulting coated nanotubes have hydroxyl groups exposed to surface. These groups react with aminopropyltriethoxysilane [APTES: (OEt)₃Si(CH₂)₃NH₂] as shown in scheme 6.

In exemplary procedures, samples A-E and uncoated nanotubes were reacted with APTES (according to the procedures described hereinabove). In the reaction, since their ordering on the coating surface was unknown, there was no guarantee that three hydroxyl groups reacted with the three ethoxy groups on APTES. A solid method for quantification of the surface groups is yet to be developed. Still, ζ potentials measured on the coated samples before and after the reaction with APTES seemed to correlate with the amount of coating.

In exemplary procedures, ζ potentials were measured on diluted ethanolic solutions of coated nanotubes before and after APTES functionalization. Before the reaction with APTES, all ζ potential values for the coated nanotubes are negative. After the reaction with APTES, the values gradually increase from less negative to increasingly positive as the coating thickness increases. This increment is a conformation to the presence of NH₂ amine groups on the composite nanotube surface. The higher the ζ potential value is, the more amine groups are on the surface. Interestingly, the maximum value for do not belong to the thickest coating. The reason to this is that as the coating gets thicker, it contours bundles of nanotubes rather than single nanotubes, lowering the surface area available for APTES reaction. Overall, ζ potential is a good analytical tool for tracking the surface functionalization of the polycarbohydrate-coated WS₂ nanotubes, and for getting an idea about their dispersability in different solvents (as illustrated in FIG. 23).

### Example 6

### Preparation of Polycarbohydrate-Engineered WS₂ Nanotubes

In exemplary procedures, Multi-walled WS₂ nanotubes (50 mg, 0.20 mmol; NanoMaterials Ltd., Batch # TWPO-MA018) were weighed into a 48 ml pressure tube (Sigma, cat. # Z568767) equipped with a small magnetic stirrer. Four ml of chloroform (Carlo Erba, ACS reagent) were added to the tube and the tube was capped and placed in a sonication bath for 5 minutes to disperse the nanotubes. A solution of glucose pentaacetate (98%, Acros, cat. #119910250) in four ml of chloroform was prepared (for saccharide and BF₃.Et₂O quantities, see Table 10). The saccharide solution was added to the tube containing the dispersed nanotubes. The overall content was cooled in an ice bath for 5 minutes while argon was bubbled into the mixture (strong bubbling). BF₃-Et₂O (Sigma, cat. #175501) was then added using a syringe and the tube was quickly capped and placed on a heating and stirring plate equipped with a Starfish™ multi-experiment workstation (Heidolph Instruments, Germany). To monitor the medium temperature (set up at 60±2° C.), an uncapped control tube with silicon oil was placed in one of the five positions of the plate.

The reaction tubes were heated and stirred for 24 h and then left for 30 minutes to cool to room temperature. Upon heating, the color of the liquid in the tubes gradually turned from colorless to yellow to dark brown, suggesting formation of saccharide decomposition products—furaldehydes. The contents of the tubes were transferred into PTFE centrifuge tubes and washed once with chloroform, to separate the suspended coated nanotubes from the non-suspended solids. Then the nanotubes were washed with EtOH absolute (Carlo Erba, ACS reagent, anhydrous) until the washing liquid was colorless and its pH was neutral. Next, two DI H₂O washes and a final wash with EtOH were done (12500 RPM, 13° C., 5 min.). The contents of the tubes were then dried overnight at 40° C. under vacuum. Table 10 presents quantitative data for the preparation of samples A-G.

TABLE 10

| | |
|---|---|
| Glucose | |
| pentaacetate | BF₃•Et₂O |

| | mg | mmol | ml | mmol |
|---|---|---|---|---|
| A | 79 | 0.20 | 0.05 | 0.53 |
| B | 157 | 0.40 | 0.1 | 1.06 |
| C | 315 | 0.81 | 0.2 | 2.13 |
| D | 472 | 1.21 | 0.3 | 3.19 |
| E | 630 | 1.61 | 0.4 | 4.26 |
| F | 787 | 2.02 | 0.5 | 5.32 |
| G | 1259 | 3.23 | 0.8 | 8.51 |

### Example 7

### Conjugation with APTES

In exemplary procedures, the samples were weighed into Eppendorf tubes and dispersed by sonication for five minutes in 2 ml EtOH. APTES (3-aminopropyltriethoxysilane, 99%, Acros cat. #15108) was then added to each tube under a stream of argon (for sample and APTES quantities, see Table 11 presenting quantitative data for APTES attachment to samples A-G and to WS₂ NTs) and the tubes were capped and shaken overnight in an orbital shaker (Intelli-Mixer RM-2L, Elmi). After 17 hours, the content was washed three times with EtOH, centrifuged (12,500 RPM, 23° C., 5 min), and then dried overnight at 40° C. in a vacuum oven.

TABLE 11

APTES

| | Sample (mg) | μL | mmol |
|---|---|---|---|
| WS₂ NTs | 4.51 | 8.51 | 0.036 |
| A | 3.50 | 6.61 | 0.028 |
| B | 5.55 | 10.47 | 0.045 |
| C | 3.75 | 7.08 | 0.030 |
| D | 3.33 | 6.28 | 0.027 |
| E | 3.97 | 7.49 | 0.032 |
| F | 2.87 | 5.42 | 0.023 |
| G | 2.26 | 4.26 | 0.018 |

### Example 8

### Magnetically Responsive INT-WS₂ Via S Affinity for Ce^{3/4+} Metal Cation/Complex-Doped Maghemite (γ-Fe₂O₃) NPs (CAN-γ-Fe₂O₃ NPs)

Ultrathin organic self-assembled monolayers (SAMs) play an important role in surface modification, and they are known to have diverse potential applications in nanotechnology. Herein, application-driven context, NPs stabilization against detrimental aggregation is a critical parameter that needs to be fully controlled by using various methods, i.e. via steric hindrance and/or charge repulsion.

In exemplary procedures, novel experimental conditions/outputs relating to the successful attachment via ligand exchange of hydrophilic chemically modified water-compatible maghemite (γ-Fe₂O₃) NPs onto inorganic nanotubes/fullerene-like nanoparticles (WS₂-INT/IFs) were performed. Indeed, such interacting CAN-γ-Fe₂O₃ nanoparticles (NPs) have been fabricated using ultrasound (US)-mediated NPs surface-doping by Ce^{3/4+} lanthanide metallic cations/complexes arising from the monoelectronic powerful oxidant CAN (ceric ammonium nitrate, Ce^{IV}(NH₄)₂(NO₃)₆). Scheme 7 presents schematic preparation of CAN-γ-Fe₂O₃ NPs nanoparticles (CAN-γ-Fe₂O₃ NPs) showing the Ce^{3/4+} cation/complex-doping phase.

In this way and during the NPs surface-doping process, the simultaneous oxidation of starting magnetite NPs to maghemite NPs together with CAN-mediated modification(s)/doping of the NPs surface resulted in the formation/obtainment of crystalline, fully hydrophilic, and strongly positively charged CAN-stabilized-γ-Fe₂O₃ NPs that formed extremely stable colloidal ddH₂O dispersions.

### Example 9

### Magnetically Responsive WS₂-INT—Surface Decoration Via S Affinity of Ce^{3/4+} Metal Cation/Complex-Doped Maghemite (γ-Fe₂O₃) NPs (CAN-γ-Fe₂O₃ NPs)

In the procedures described below, the high-power ultrasonication allowed non-aggregated super-paramagnetic Ce^{3/4+} metal cation-doped maghemite (γ-Fe₂O₃) NPs (CAN γ-Fe₂O₃ NPs; Scheme 7). Without being bound by any particular theory, it is assumed that (i) ultrasonication allows a much higher and robust/reproducible level of Ce^{3/4+} cation/complex surface doping and (ii) that doping Ce^{3/4+} cations/complexes [Ce^{3/4+}Lₙ] (Lₙ: Ce atom ligand) act as “hard” Lewis acid centers exploiting the known rich [Ce^{3/4+}]-complex ligand coordination/exchange chemistry. This improved CAN-mediated doping process was made quantitatively significant relating to the amount of doping Ce^{3/4+} cations/complexes [Ce^{3/4+}Lₙ] that resulted in an innovative chemical way to bind any Lewis-base-containing species (N/O/S-heteroatom-containing organic moieties) for an NP decoration/functionalization.

### CAN-γ-Fe₂O₃ NPs Decoration onto WS₂ Inorganic Nanotubes (WS₂ INTs) and/or Fullerene-Like Nanoparticles WS₂ IFs

In exemplary procedures, various amounts of CAN-γ Fe₂O₃ NPs was added into different vials, each vial containing 5.0 mg of either INTs or IFs suspended in 5.0 mL of H₂O. The vials were then placed in an ultrasonic bath for 7 minutes while different amounts of CAN-γ-Fe₂O₃ NPs were added to vials. The vials were then placed into an orbital shaker for varying intervals. See Table 12 below presenting the corresponding data, presenting the amounts of CAN-γ-Fe₂O₃ NPs vs. 50 mg of INTs suspended in 3 mL of H₂O.

Purification of Related Magnetic Composites INTs/IFs—CAN-γ-Fe₂O₃ NPs:

In exemplary procedures, the resultant products of the interaction Ce-ligand exchange reaction involving Ce^{3/4+} cation/complex-doped NPs, i.e. INTs/IFs-CAN-γ-Fe₂O₃ NPs were then separated from the solution by attaching the reaction vial to a 0.5 T external magnet for washing steps. The NPs-coated INTs/IFs-CAN-γ-Fe₂O₃ NPs composites were drawn immediately towards the magnet as oppose to uncoated INT/IF and CAN-γ-Fe₂O₃ NPs. The sediment was washed several times (×5, H₂O). The resulting sediment of each vial was freeze-dried overnight in a lyophilyzer.

Scheme 8 presents isolation of INTs/IFs-CAN-γ-Fe₂O₃ NPs composites from experiment solutions via magnet attractive forces apart from starting CAN-maghemite NPs and non-magnetic INTs/IFs that were not attached by magnetic CAN-γ-Fe₂O₃ nanoparticles.

TABLE 12

| | | Ratio of | | | |
|---|---|---|---|---|---|
| CAN-γ- | Fe | INTs to | Reaction | Size | Zeta |
| Fe₂O₃ NPs* | (aq) | CAN-γ- | duration | Distribution | Potential |
| (mL) | (mg) | Fe₂O₃ NPs | (hrs) | (nm) | (mV) |
| 0 | 0 | — | | 613.7 ± 4.336 | −30.2 ± |
| | | | | | .404 |
| 1.39 | 5.00 | 10 | 3 | 3379 ± 113.8 | 15.5 ± |
| | | | | | 0.1 |
| 1.39 | 5.00 | 10 | 6 | 2652 ± 207.5 | 11.1 ± |
| | | | | | 0.361 |
| 1.39 | 5.00 | 10 | 18 | 797.3 ± 79.41 | 22.8 ± |
| | | | | | 0.208 |
| 2.79 | 10.0 | 5 | 3 | 2350. ± 104.5 | 21.3 ± |
| | | | | | 0.208 |
| 2.79 | 10.0 | 5 | 6 | 3155 ± 164 | 18.3 ± |
| | | | | | 1.1 |
| 2.79 | 10.0 | 5 | 18 | 953.1 ± 0.57 | 21.6 ± |
| | | | | | 0.173 |
| CAN-γ- | — | — | — | 45.91 ± 0.200 | 33.0 ± |
| Fe₂O₃ | | | | | 1.20 |

*3.59 mg/mL

Thus when starting WS₂ INTs have a negative value of zeta potential, −30.2±0.4 mV, while CAN-γ-Fe₂O₃ NPs have a positive zeta potential value of 33.0±1.2 mV. In this experiment, two weight ratios of CAN-γ-Fe₂O₃ NPs and WS₂ INTs were chosen to explore the feasibility and weight optimal amounts between both reactive materials.

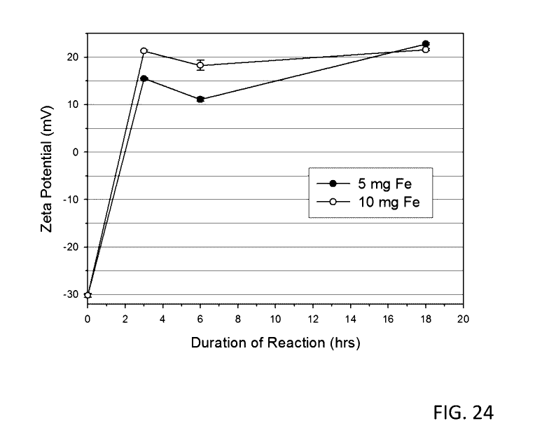

FIG. 24 presents zeta Potential (mV) of different ratios of INTs to CAN-maghemite NPs at varying duration of attachment reaction (3, 6, 18 hrs).

Chemically modified WS₂ INTs at higher concentrations have higher zeta potential values (15.5±0.1 and 21.3±0.2 mV respectively). Nevertheless, values differences were not so significant since it appears that the NPs coating/attachment reaches saturation after three hours contact reaction. Zeta potential changes afterwards were mild and not more significant.

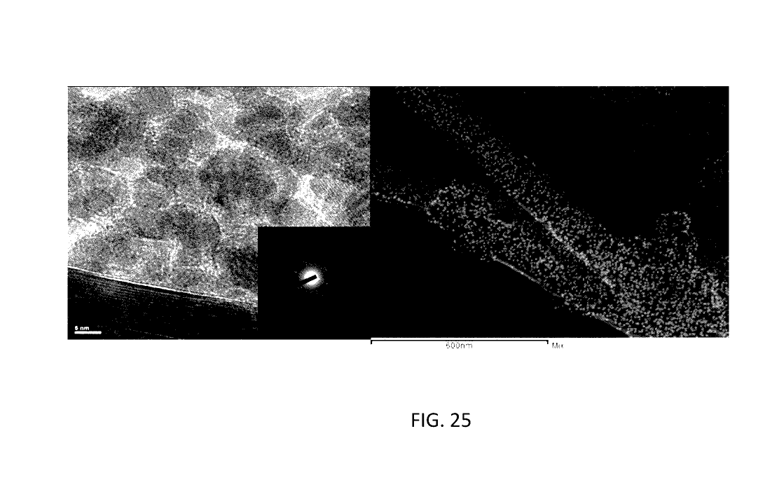

FIG. 25 presents HRTEM image of INTs/IFs-CAN-γ-Fe₂O₃ NPs composites and selected area electron diffraction (SAED) RX diffraction of NPs-coated WS₂ INTs.

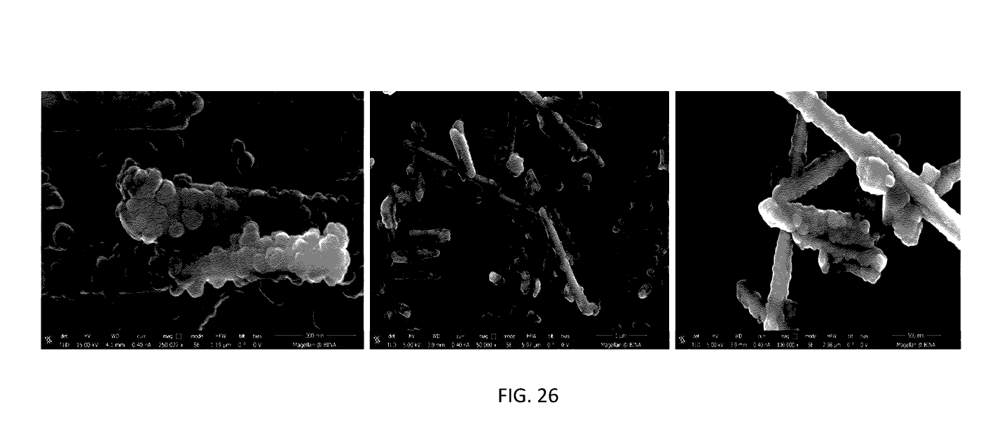

FIG. 26 presents HRSEM images of CAN-γ-Fe₂O₃ NPs attached onto WS₂-INTs.

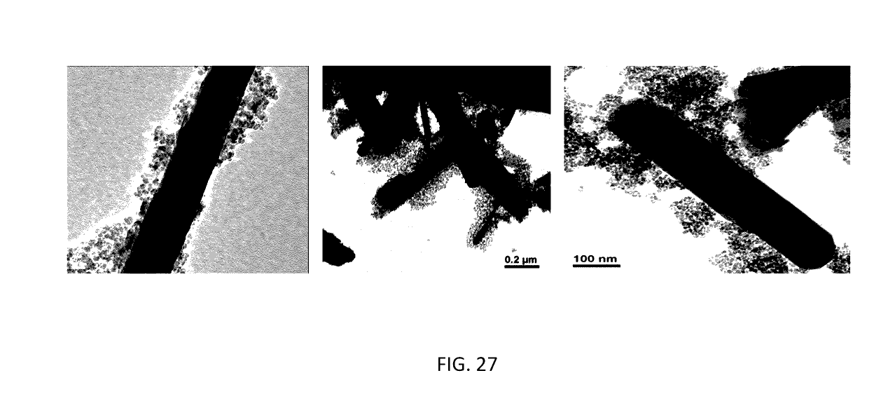

FIG. 27 presents HRTEM and TEM images of CAN-γ-Fe₂O₃ attached onto WS₂-INTs.

According to HRTEM pictures, inorganic INTs were almost completely coated with CAN-γ-Fe₂O₃ nanoparticles. The thickness of the coating might be non-uniform since the disclosed experimental conditions secured the formation of monolayers as well as multilayers of NPs onto chemically modified WS₂ INTs.

From both HRTEM and HRSEM images, it appears that INTs might be fully coated by chemically reactive CAN-γ-Fe₂O₃ NPs when using the described contact reaction procedure. As a main attractive output of such a NPs-mediated decoration beyond interesting magnetic properties of related composite materials, one might achieve such a NPs-mediated functionalization of the INTs surface via controlled modulation of NPs-based layering steps, meaning getting underlayered (much thin NPs coating), monolayered and even multilayered deposited NPs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A hybrid inorganic-organic composite comprising a nanostructure of a metal chalcogenide and at least one compound comprising an organic cyclic moiety, wherein said compound is linked to said nanostructure.

2. The hybrid inorganic-organic composite of claim 1, wherein said cyclic moiety is a heterocyclic moiety.

3. The hybrid inorganic-organic composite of claim 1, wherein said metal chalcogenide is metal dichalcogenide.

4. The hybrid inorganic-organic composite of claim 1, wherein said compound is covalently linked to said nanostructure.

5. The hybrid inorganic-organic composite of claim 1, wherein said nanostructure is in the form of a nanotube.

6. The hybrid inorganic-organic composite of claim 1, wherein said nanostructure is in the form of fullerene.

7. The hybrid inorganic-organic composite of claim 1, wherein said compound has the structure of Formula I: 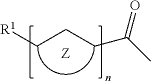
such that said hybrid composite has the structure of Formula II: 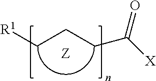
wherein:
Z represents a cyclic or heterocylic moiety;
n is an integer of from 1 to 100;
X represents a metal chalcogenide or a metal dichalcogenide selected from the group consisting of: TiS₂, TiSe₂, TiTe₂, WS₂, WSe₂, WTe₂, MoS₂, MoSe₂, MoTe₂, SnS₂, SnSe₂, SnTe₂, RuS₂, RuSe₂, RuTe₂, GaS, GaSe, GaTe, InS, InSe, HfS₂, ZrS₂, VS₂, ReS₂ or NbS₂; and
R₁ represents or comprises hydrogen, any polymer and/or organic moiety selected from the group consisting of: hydrogen, alkyl, cycloalkyl, aryl, heterocyclic, heteroaryl, alkoxy, hydroxy, thiohydroxy, thioalkoxy, aryloxy, thioaryloxy, aniline, amino, nitro, halo, trihalomethyl, cyano, amide, carboxyl, sulfonyl, sulfoxy, sulfinyl, sulfonamide, and a saccharide.

8. The hybrid inorganic-organic composite of claim 7, wherein said compound comprising a cyclic moiety covalently is linked to said nanostructure via S or Se atom linkage.

9. The hybrid inorganic-organic composite of claim 7, being in the form of Formula III: 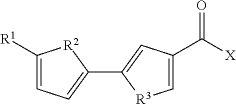
wherein R² and R³ are each, independently, selected from the elements S and O.

10. The hybrid inorganic-organic composite of claim 7, wherein R¹ is represented by Formula IV: 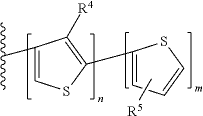
wherein n and m are each independently an integer of from 0 to 100;
R⁴ comprises or is selected from the group consisting of: hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, and a saccharide; and
R⁵ comprises or is selected from the group consisting of hydrogen, alkyl, cycloalkyl, aryl, heteroalicyclic, heteroaryl, alkoxy, hydroxy, thiohydroxy, thioalkoxy, aryloxy, thioaryloxy, amino, nitro, halo, trihalomethyl, cyano, amide, carboxy, sulfonyl, sulfoxy, sulfinyl, sulfonamide, a saccharide or is a fused ring.

11. The hybrid inorganic-organic composite of claim 7, having a structure represented by Formula V:

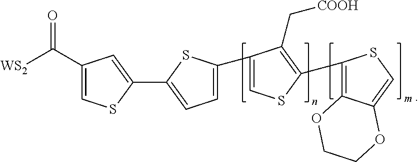

12. The hybrid inorganic-organic composite of claim 1, being in the form of a core-shell, wherein said core is or comprises a plurality of said metal chalcogenide and said shell is or comprises a plurality of said cyclic moiety.

13. The hybrid inorganic-organic composite of claim 12, wherein an outside surface of said shell is characterized by a layer selected from a polyCOOH, polyOH, and chemical derivative thereof.

14. The hybrid inorganic-organic composite of claim 1, wherein said cyclic moiety is or comprises a saccharide-related moiety and wherein said nanostructure is attached to an anomeric carbon of said saccharide moiety.

15. The hybrid inorganic-organic composite of claim 1, wherein said cyclic moiety is a polycyclic moiety.

16. A process for producing a hybrid composite, comprising the steps of:
mixing bithiophene or a derivative thereof and a dehydrating agent is selected from POCl₃, PCl₃, SOCl₂, SO₂Cl₂, SO₃, PCl₅, P₂O₅, VOCl₃, AlCl₃, TiCl₄, acetic anhydride, trifluoromethanesulfonic anhydride thereby synthesizing 2,2′-bithiophene-5-carboxaldehyde; and
adding to said 2,2′-bithiophene-5-carboxaldehyde a nanostructure of metal chalcogenide or a metal dichalcogenide, thereby producing said hybrid composite.

17. A nanotube of metal chalcogenide nanostructure having attached on at least one surface thereof one or more chemically reactive nanoparticles.

18. The nanotube of claim 17, wherein said chemically reactive nanoparticles are Fe₂O₃ nanoparticles.

19. The nanotube of claim 17, further comprising cerium cations.

20. The nanotube of claim 17, being in the form of a core-shell, wherein said core comprises a metal chalcogenide and said shell comprises said metal cation or complex-doped maghemite γ-Fe₂O₃, and optionally further comprises cerium ions.
